(19) United States
(12) Patent Application Publication
(10) Pub. No.: US 2024/0188904 A1
(43) Pub. Date: Jun. 13, 2024

(54) CONTINUOUS GLUCOSE MONITORING SYSTEM INSIGHT NOTIFICATIONS

(71) Applicant: Dexcom, Inc., San Diego, CA (US)

(72) Inventors: Afshan A. KLEINHANZL, San Diego, CA (US); Nicholas Alexander FRATI, San Diego, CA (US); Kerry Roller JOHNSON, San Diego, CA (US); James LO, San Diego, CA (US); Stacey Lynne FISCHER, San Diego, CA (US); Barbara Weronika SEKUDEWICZ, San Diego, CA (US); Mackenna Elizabeth LEES, San Diego, CA (US); John E. GOODE, San Deigo, CA (US)

(21) Appl. No.: 18/538,982

(22) Filed: Dec. 13, 2023

Related U.S. Application Data
(60) Provisional application No. 63/387,269, filed on Dec. 13, 2022.

(51) Int. Cl. A61B 5/00; A61B 5/145

(52) U.S. Cl. CPC A61B 5/7435; A61B 5/002; A61B 5/14532; A61B 5/7282; A61B 5/7475; A61B 5/681

(57) ABSTRACT

Certain embodiments provide a system that generates and presents contemporaneous insight notifications on a display device. The contemporaneous insight notifications are based on measured analyte data provided by a sensor device worn by a user. The insight notifications help the user determine why analyte level fluctuations may be contemporaneously occurring, such as how the user's recent meals, activities, etc., may have impacted the user's measured analyte concentration levels.

### CROSS-REFERENCE TO RELATED APPLICATIONS

This application claims the benefit of U.S. Provisional Application Ser. No. 63/387,269 (filed on Dec. 13, 2022), the contents of which are incorporated by reference herein in its entirety.

### BACKGROUND

Generally, a continuous glucose monitoring (CGM) system may include a CGM sensor device worn by a user and a CGM display device (or “display device”). The CGM sensor device includes a glucose sensor and a sensor electronics module with a processor and a wireless transceiver. The display device is a mobile computing device with a processor and a wireless transceiver, such as a CGM data receiver, a smartphone, a smartwatch, a tablet computer, a laptop computer, etc. The CGM sensor device measures the user's glucose concentration levels, generates measured glucose data, and transmits the measured glucose data to the display device for presentation to the user in a graphical user interface (GUI).

Patients with prediabetes, type 1 diabetes (insulin treated), type 2 diabetes (insulin treated or non-insulin treated), as well as the general population (such as health and longevity users, etc.), may use a CGM sensor device to measure their glucose concentration levels during the day, such as every 1 minute, 5 minutes, 10 minutes, etc. The CGM sensor device may periodically transmit the measured glucose data to the display device (such as every 5 minutes, 10 minutes, 30 minutes, 60 minutes, etc.). The CGM sensor device may also transmit the measured glucose data in response to a request from the display device. After receiving the user's measured glucose data for an extended period of time, such as 10 days, 2 weeks, etc., the display device may provide a retrospective analysis of the measured glucose data that is intended to help the user determine how the user's meals, activities, etc. may have impacted the user's glucose concentration levels over the extended period of time.

Unfortunately, people tend to remember only those meals, activities, etc. that happened very recently, such as the same day, the previous day, or within the past few days. Accordingly, a retrospective analysis of the user's glucose concentration levels after an extended period of time may not help the user pinpoint the particular meals and activities that may have impacted the user's glucose levels. Additionally, people may become anxious, frightened, or even panicky when the display device presents a contemporaneous indication (such as a trend arrow in a GUI) that the user's glucose concentration level is rising without any further comment, explanation, or elucidation.

### BRIEF DESCRIPTION OF THE DRAWINGS

FIG. 1 illustrates aspects of an example health management system, in accordance with embodiments of the present disclosure.

FIGS. 2A, 2B, and 2C illustrate aspects of an example continuous analyte monitoring (CAM) system, in accordance with embodiments of the present disclosure.

FIG. 3 illustrates example input data and metric data for use by the health management system of FIG. 1, in accordance with embodiments of the present disclosure.

FIG. 4 depicts a block diagram of an example computer device, in accordance with embodiments of the present disclosure.

FIGS. 5A, 5B, 5C depict exemplary analyte rapidly rising insight notifications, in accordance with embodiments of the present disclosure.

FIGS. 6, 7 depict exemplary analyte spike insight notifications, in accordance with embodiments of the present disclosure.

FIG. 8 depicts an exemplary analyte falling insight notification, in accordance with embodiments of the present disclosure.

FIG. 9 depicts an exemplary analyte back-in-range insight notification, in accordance with embodiments of the present disclosure.

FIGS. 10, 11 depict exemplary analyte time-in-range insight notifications, in accordance with embodiments of the present disclosure.

FIGS. 12, 13 depict exemplary meal related event notifications, in accordance with embodiments of the present disclosure.

FIGS. 14A, 14B depict exemplary related event notifications, in accordance with embodiments of the present disclosure.

FIG. 15 depicts an example process flow diagram for providing insight notifications on a CGM display device, in accordance with embodiments of the present disclosure.

### DETAILED DESCRIPTION

Embodiments of the present disclosure advantageously generate and present contemporaneous insight notifications on the display device based on the measured analyte data provided by the sensor device worn by the user. The insight notifications help the user determine why analyte level fluctuations may be contemporaneously occurring, such as how the user's recent meals, activities, etc., may have impacted the user's measured analyte concentration levels. Generally, embodiments of the present disclosure may be applied to many diseases and their associated analytes, as well as to users who do not have a disease but would like to understand the effect of various analyte concentration levels on their bodies (such as glucose, etc.). For example, the disease may be diabetes and the analyte may be glucose.

The display device may analyze the user's measured analyte data over a predetermined time period, such as the past 5 minutes, 10 minutes, 30 minutes, the past hour, the past 3 hours, etc., in order to identify insight events and provide real time or near-real time insight notifications to the user. Advantageously, due to the contemporaneous and expressive nature of these insight notifications, the user may immediately explore various mitigation techniques, such as drinking water, go for a walk, simply waiting it out, etc. The display device may also analyze the user's measured analyte data over a somewhat longer predetermined time period, such as the current day, the past day, the past several days, the past 5 days, the past 7 days, etc., in order to identify insight events and provide relatively contemporaneous insight notifications.

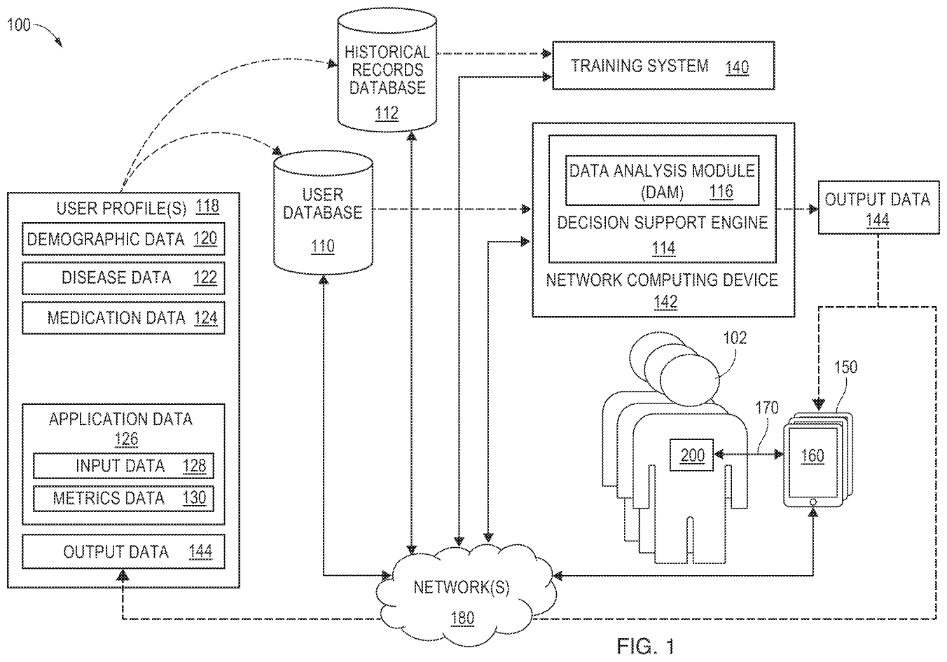

FIG. 1 illustrates aspects of health management system 100, in accordance with embodiments of the present disclosure.

Generally, health management system 100 provides decision support (e.g., diagnosis or lack of a diagnosis, insights, treatment recommendations, and/or the like) to each user 102 based on measured analyte data acquired by CAM system 200 worn by each user 102.

In certain embodiments, health management system 100 includes, inter alia, user database 110, historical records database 112, training system 140 connected to network(s) 180, network computing device 142 connected to network(s) 180, mobile computing devices (or display devices) 150 connected to network(s) 180, and CAM systems 200. Network(s) 180 may include one or more local area networks (LANs), wireless LANs (WLANs), low power wide area networks (LPWANs), wide area networks (WANs), cellular networks (such as 3G, 4G, LTE, 5G, 6G, etc.), the Internet, etc., employing various network topologies and protocols (hereinafter “network 180”). For example, network 180 may also include various combinations of wired and/or wireless physical layers, such as, for example, copper wire or coaxial cable networks, fiber optic networks, WiFi networks, Bluetooth mesh networks, CDMA, FDMA and TDMA cellular networks, etc.

User database 110 may be hosted by a network database server connected to network 180; alternatively, user database 110 may be hosted by network computing device 142 (indicated by a dashed line). User database 110 may store user profile 118 for each user 102 which may include, inter alia, demographic data 120, disease data 122, medication data 124, application data 126 including input data 128 (such as measured analyte data) and metric data 130, and output data 144 (such as a disease prediction). Similarly, historical records database 112 may be hosted by a network database server connected to network 180; alternatively, historical records database 112 may be hosted by training system 140 (indicated by a dashed line). Historical records database 112 may store, inter alia, historical analyte data and historical outcome data associated with the historical analyte data. The historical outcome data may include, inter alia, clinical disease diagnoses that indicate whether each user of the population has been clinically diagnosed with the particular disease based on one or more independent sources, such as sources that did not consider the historical analyte data.

Training system 140 is configured to evaluate, select and train disease prediction models in accordance with embodiments of the present disclosure. Training system 140 may include one or more network computing devices.

Network computing device 142 is configured to store and execute decision support engine (DSE) 114, as well as other software modules, applications, etc., to perform certain functionality described below. DSE 114 may include, inter alia, data analysis module (DAM) 116, as well as other software modules. In certain embodiments, training system 140 may include network computing device 142.

Display devices 150 are configured to store and execute one or more software applications that present one or more GUIs 160 to display certain data including, inter alia, input data 128 (such as measured analyte data, etc.), output data 144, etc. In certain embodiments, at least a portion of DSE 114 and DAM 116 may be stored and executed by display device 150.

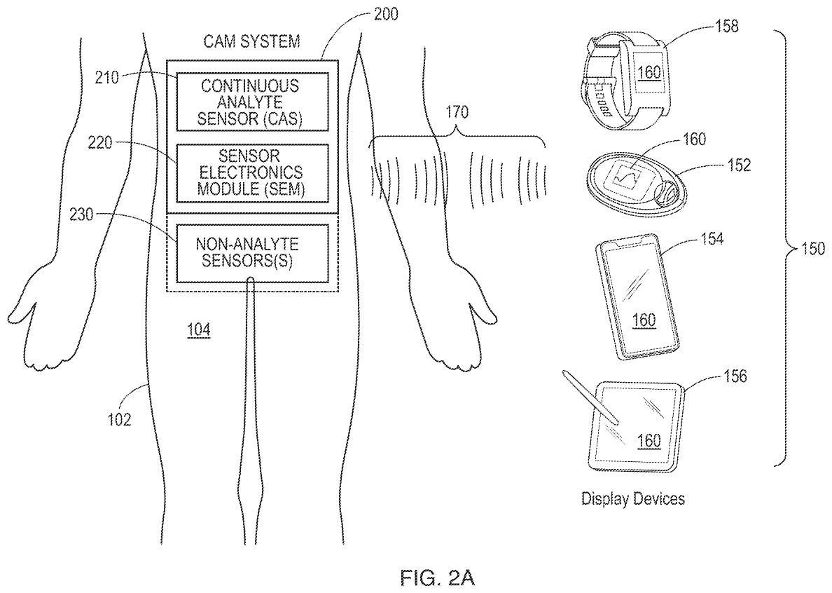

CAM systems 200 are configured to operate continuously to monitor one or more analytes for users 102. Each CAM system 200 is worn by a user 102, and may be coupled to a display device 150 via wireless connection 170 to transfer measured analyte data (and other data) to display device 150. Wireless connection 170 may be a Bluetooth connection, a Bluetooth Low Energy (BLE) connection, an RFID or NFC connection, an IEEE 802.11 connection (Wi-Fi), etc. CAM system 200 is described in more detail with respect to FIGS. 2A, 2B, 2C.

The term “analyte” as used herein is a broad term used in its ordinary sense, including, without limitation, to refer to a chemical substance, compound, molecule, element, etc., in a biological fluid (such as blood, interstitial fluid, cerebral spinal fluid, lymph fluid, urine, etc.) that may be identified or measured, and analyzed.

Analytes may include naturally occurring substances, artificial substances, pharmacologic agents, metabolites, ions, blood gasses, hormones, neurotransmitters, vitamins, minerals, peptides, pathogens, toxins, and/or reaction products. Analytes for measurement by the devices and methods of the present disclosure may include (but may not be limited to) glucose; lactate; potassium; troponin; creatinine; ketone; acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; creatinine phosphokinase (CPK); cyclosporin A; cystatin C; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids may also constitute analytes in certain implementations. Ions are a charged atoms or compounds that may include the following (sodium, potassium, calcium, chloride, nitrogen, or bicarbonate, for example). The analyte may be naturally present in the biological fluid, for example, a metabolic product, a hormone, an antigen, an antibody, an ion etc. Alternatively, the analyte may be introduced into the body or exogenous, for example, a contrast agent for imaging, a radioisotope, a chemical agent, a fluorocarbon-based synthetic blood, a challenge agent analyte (such as introduced for the purpose of measuring the increase and or decrease in rate of change in concentration of the challenge agent analyte or other analytes in response to the introduced challenge agent analyte), or a drug or pharmaceutical composition, including but not limited to exogenous insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine The metabolic products of drugs and pharmaceutical compositions are also contemplated analytes. Analytes such as neurochemicals and other chemicals generated within the body may also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

In certain embodiments, CAM system 200 is configured to continuously measure one or more analytes and transmit the measured analyte data to an electric medical records (EMR) system (not shown in FIG. 1). An EMR system includes one or more network computing devices that host a software platform that is configured to receive, store and manage medical data. An EMR system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system may also be used to create reports for clinical care and/or disease management for a patient. In certain embodiments, the EMR system may be in communication with network computing device 142 over network 180 to perform certain techniques described herein. In other embodiments, an EMR system may provide access to population-level health statistics, health economics, and the generation of clinical evidence or assessment of healthcare outcomes. In particular, as described herein, DSE 114 may access the EMR system to obtain data associated with a user 102, such as measured analyte data, for disease prediction purposes. In some cases, DSE 114 may provide the disease prediction to the EMR system.

CAM system 200 is configured to continuously measure one or more analyte concentration levels, and then transmit measured analyte data to display device 150 over wireless connection 170. In certain embodiments, a single-analyte sensor may be configured to generate an analog sensor signal that is proportional to the concentration level of a respective analyte, and a sensor electronics module may be configured to sample the analog sensor signal, generate measured analyte data, and transmit the measured analyte data to a display device 150. In certain embodiments, CAM system 200 periodically transmits the measured analyte data to display device 150 during the wear session. In other embodiments, CAM system 200 stores the measured analyte data in a memory, and transmits the measured analyte data to display device 150 at the conclusion of the wear session.

In certain embodiments, CAM system 200 may include multiple single-analyte sensors, and each single-analyte sensor generates an analog sensor signal that is proportional to the concentration level of a particular analyte. In other embodiments, CAM system 200 may include a multi-analyte sensor that generates multiple analog sensor signals, and each analog sensor signal is proportional to the concentration level of a particular analyte. In further embodiments, CAM system 200 may include multiple multi-analyte sensors, a combination of single-analyte sensors and multi-analyte sensors, etc.

In certain embodiments, CAM system 200 may transmit the measured analyte data directly to network computing device 142 via network 180 for review, retrieval, execution of further analytics, etc. In such embodiments, CAM system 200 may be equipped with a mobile internet of things (IoT) interface, such as an LPWAN transceiver (such as LTE-M, Cat-M1, NB-IoT, etc.), a cellular radio transceiver, a Wi-Fi transceiver, etc., to transmit the measured analyte data over network 180.

Display devices 150 may be mobile computing devices that are wirelessly connected to network 180, using a WLAN, a cellular network, etc. In certain embodiments, display devices 150 may include a CAM data receiver, a smartphone, a tablet computer, a smartwatch, a laptop computer, etc. In some embodiments, display device 150 may transmit the measured analyte data to one or more other individuals having an interest in the health of the patient (such as a family member or physician for real-time treatment and care of the patient).

Generally, display device 150 is configured to receive and process measured analyte data from CAM system 200, and may store and execute one or more applications, such as a mobile health application, etc. In particular, display device 150 may store information about a user, including the user's measured analyte data, in a user profile 118 that is associated with the user. These data may be stored by display device 150 as well as user database 110.

Generally, DSE 114 may include one or more software modules, such as DAM 116, etc. In certain embodiments, DSE 114 may be stored and executed by network computing device 142, which communicates with display device 150 over network 180. In other embodiments, the software modules (or relevant functionality) may be distributed across multiple devices, and a portion of DSE 114 may be stored and executed by display device 150 and/or CAM system 200, while the remaining portion of DSE 114 may be stored and executed by network computing device 142. In some other embodiments, DSE 114 may be stored and executed by display device 150 and/or CAM system 200. Generally, DSE 114 may generate insight notifications for display on display device 150 based on the measured analyte data. In certain embodiments, DSE 114 may provide decision support recommendations based on information included in user profile 118.

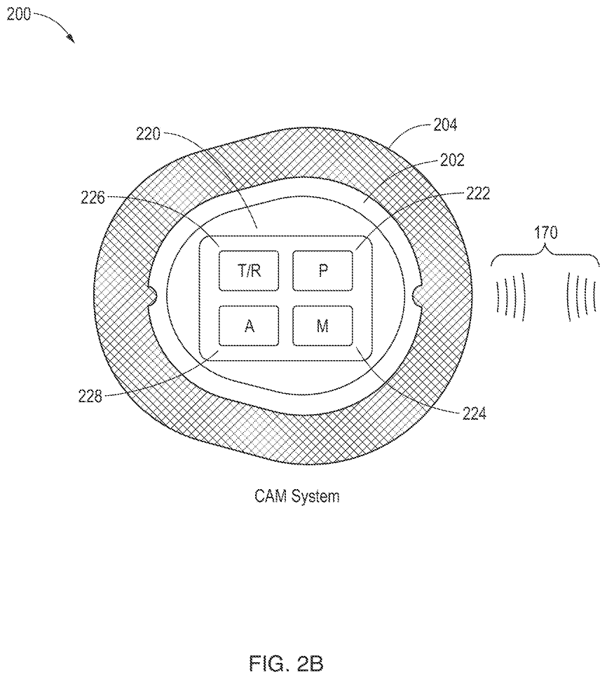

User profile 118 may include information collected about the user. For example, display device 150 may collect and store input data 128, including the measured analyte data received from CAM system 200, in user profile 118. In certain embodiments, input data 128 may include other data in addition to measured analyte data received from CAM system 200. For example, additional input data 128 may be acquired through manual user input, one or more other non-analyte sensors or devices, various processes executing on display device 150, etc. Input data 128 of user profile 118 are described in further detail below with respect to FIG. 3.

DAM 116 may be configured to generate metric data 130 based on input data 128. Metric data 130, discussed in more detail below with respect to FIG. 3, are generally indicative of the health or state of a user, such as one or more of the user's physiological state, trends associated with the health or state of a user, analyte features, etc. In certain embodiments, DSE 114 may provide decision support to a user based on metric data 130 and/or input data 128. As shown, metric data 130 are also stored in user profile 118.

User profile 118 also includes demographic data 120, disease data 122, and/or medication data 124 (such as type of medication, brand of medication, dosage, frequency of administration). In certain embodiments, such information may be provided through user input or obtained from certain data sources (such as electronic medical records, EMR systems, etc.). In certain embodiments, demographic data 120 may include one or more of the user's age, body mass index (BMI), ethnicity, gender, etc. In certain embodiments, disease data 122 may include information about a condition of a user, such as whether the user has been previously diagnosed with or experienced various diseases, such as diabetes, liver disease, kidney disease, heart disease, hyperglycemia, hypoglycemia, co-morbidities, etc. In certain embodiments, information about a user's condition may also include the length of time since diagnosis, the level of control, level of compliance with condition management therapy, other types of diagnosis (such as heart disease, obesity) or measures of health (such as heart rate, exercise, stress, sleep, etc.), and/or the like.

In certain embodiments, medication data 124 may include information about the amount, frequency, and type of a medication taken by a user. In certain embodiments, the amount, frequency, and type of a medication taken by a user is time-stamped and correlated with the user's analyte levels, thereby, indicating the impact the amount, frequency, and type of the medication had on the user's analyte levels.

In certain embodiments, user profile 118 may be dynamic because at least part of the information that is stored in user profile 118 may be revised over time and/or new information may be added to user profile 118 by DSE 114, display device 150, etc. Accordingly, information in user profile 118 stored in user database 110 may provide an up-to-date repository of information related to a user.

User database 110 may be implemented as any type of data store, such as relational databases, non-relational databases, key-value data stores, file systems including hierarchical file systems, etc. In some embodiments, user database 110 may be distributed. For example, user database 110 may comprise persistent storage devices, which are distributed. Furthermore, user database 110 may be replicated so that the storage devices are geographically dispersed.

Similarly, historical records database 112 may be implemented as any type of data store, such as relational databases, non-relational databases, key-value data stores, file systems including hierarchical file systems, etc. In some embodiments, historical records database 112 may be distributed. For example, historical records database 112 may comprise persistent storage devices, which are distributed. Furthermore, historical records database 112 may be replicated so that the storage devices are geographically dispersed.

Although depicted as separate databases for conceptual clarity, in some embodiments, user database 110 and historical records database 112 may be combined into a single database. In other words, the historical and current data related to users of CAM system 200, as well as historical data related to patients that were not previously users of CAM system 200, may be stored in a single database.

User database 110 may include user profiles 118 associated with a number of users who similarly interact with respective display devices 150. User profiles 118 stored in user database 110 may be accessible over network 180. As described above, DSE 114, and more specifically DAM 116, may fetch input data 128 from user database 110 and generate metric data 130 which may then be stored as application data 126 in user profile 118.

In certain embodiments, user profiles 118 stored in user database 110 may also be stored in historical records database 112. User profiles 118 stored in historical records database 112 may provide a repository of up-to-date information and historical information for each user. Thus, historical records database 112 essentially provides all data related to each user of CAM system 200. In certain embodiments, the data may be stored with an associated timestamp to identify when information related to a user has been obtained, updated, etc.

Further, historical records database 112 may maintain time series data collected for users over a period of time (such as 5 years), including for users who use CAM system 200. Further, in certain embodiments, historical records database 112 may also include data for one or more patients who are not users of CAM system 200. For example, historical records database 112 may include information (such as user profiles) related to one or more patients treated by a healthcare physician. Data stored in historical records database 112 may be referred to herein as population data.

Data related to each patient stored in historical records database 112 may provide time series data collected over a disease lifetime of the patient. For example, the data may include information about the patient prior to being diagnosed and information associated with the patient during the lifetime of the treatment, including information related to level of treatment required, as well as information related to other diseases or conditions. Such information may indicate symptoms of the patient, physiological states of the patient, measured analyte data for the patient, states/conditions of one or more organs of the patient, habits of the patient (such as activity levels, food consumption, etc.), medication prescribed, etc., throughout the lifetime of the treatment.

FIG. 2A depicts a diagram of CAM system 200 and display devices 150, in accordance with embodiments of the present disclosure.

In certain embodiments, CAM system 200 includes, inter alia, continuous analyte sensor (CAS) 210, sensor electronic module (SEM) 220, and a power source, such as a battery. One or more non-analyte sensors (NAS) 230 or other devices may also be coupled to SEM 220.

Generally, CAS 210 may include one or more single-analyte sensors, one or more multi-analyte sensors, a combination of single-analyte sensors and multi-analyte sensors, etc. Each single-analyte sensor generates an analog sensor signal that is proportional to the concentration level of a particular analyte. Similarly, each multi-analyte sensor generates multiple analog sensor signals, and each analog signal is proportional to the concentration level of a particular analyte. As an illustrative example, CAS 210 may include a single-analyte sensor configured to measure glucose concentration levels, and one or more multi-analyte sensors configured to measure lactate concentration levels, potassium concentration levels, troponin concentration levels, creatinine concentration levels, etc. As another illustrative example, CAS 210 may include a multi-analyte sensor configured to measure glucose concentration levels, lactate concentration levels, potassium concentration levels, troponin concentration levels, creatinine concentration levels, etc.

Accordingly, CAS 210 is configured to generate at least one analog sensor signal that is proportional to the concentration level of particular analyte, and SEM 220 is configured to sample the analog sensor signal, generate measured analyte data, and transmit the measured analyte data to display device 150 via wireless connection 170. SEM 220 is configured to sample the analog sensor signal at a particular sampling period (or rate), such as every 1 second (1 Hz), 5 seconds, 10 seconds, 30 seconds, 1 minute, 3 minutes, 5 minutes, etc., and to transmit the measured analyte data to display device 150 at a particular transmission period (or rate), which may be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte data transmitted to display device 150 include at least one analyte concentration level measurement having an associated time tag, sequence number, etc.

CAS 210 may be a non-invasive device, a subcutaneous device, a transcutaneous device, a transdermal device, a dermal device, an intradermal device, a subdermal device, an intravascular device, etc. In certain embodiments, CAS 210 may be configured to continuously measure analyte concentration levels using one or more measurement techniques, such as enzymatic, immunometric, aptameric, amperometric, voltametric, potentiometric, impedimetric, conductimetric, chemical, physical, electrochemical, spectrophotometric, polarimetric, calorimetric, iontophoretic, radiometric, immunochemical, optical, ion-selective, etc.

Display devices 150 may be mobile computing devices that are connected network 180. In certain embodiments, display devices 150 may include CAM data receiver 152, smartphone 154, tablet computer 156, smartwatch 158, laptop computer (not shown), etc. In some embodiments, display devices 150 may be non-mobile computing devices (such as a desktop computer, etc.) that are connected to network 180.

In certain embodiments, display devices 150 are configured for displaying data, including measured analyte data, which may be transmitted by SEM 220. Display devices 150 may include a touchscreen display for displaying data to a user and receiving inputs from the user. For example, GUI 160 may be presented to the user for such purposes. In some embodiments, display devices 150 may include other types of user interfaces such as a voice user interface instead of, or in addition to, a touchscreen display for communicating data to the user of display device 150 and receiving user inputs.

In some embodiments, one, some, or all of display devices 150 are configured to display or otherwise communicate the data as it is communicated from SEM 220 (such as in a data package that is transmitted to respective display devices 150), without any additional prospective processing required for calibration and real-time display of the data. In certain embodiments, the display devices 150 may be configured for providing alerts/alarms/notifications based on the displayable data.

For example, CAM data receiver 152 may be a custom display device specially designed for displaying certain types of data associated with measured analyte data received from SEM 220. For another example, smartphone 154 may use a commercially available operating system (OS), and may be configured to display a graphical representation of the continuous measured analyte data (such as including current and historic data) using GUI 160.

Because different display devices 150 provide different user interfaces, the content of the data packages (such as amount, format, and/or type of data to be displayed, alarms, etc.) may be customized (such as programmed differently by the manufacture and/or by an end user) for each particular display device 150. Accordingly, in certain embodiments, a number of different display devices 150 may be in direct wireless communication with a SEM 220 of a CAM system 200 worn by a user 102 during a wear session to enable a number of different types and/or levels of display and/or functionality associated with the displayable data. In certain embodiments, the type of alarms customized for each particular display device 150, the number of alarms customized for each particular display device 150, the timing of alarms customized for each particular display device 150, and/or the threshold levels configured for each of the alarms (such as for triggering) are based on output data 144.

NAS 230 may include a temperature sensor, an altimeter sensor, an accelerometer sensor, a respiration rate sensor, a sweat sensor, a heart rate sensor, an electrocardiogram (ECG) sensor, a blood pressure sensor, a respiratory sensor, an oxygenated hemoglobin sensor (spO₂), etc. Other devices may be coupled to SEM 220, such as an insulin pump, a peritoneal dialysis machine, a hemodialysis machine, etc.

FIGS. 2B, 2C depict top and side views of CAM system 200, respectively, in accordance with embodiments of the present disclosure.

CAM system 200 includes housing 202 enclosing SEM 220, and adhesive pad 204 disposed on the bottom surface of housing 202. CAS 210 protrudes from the bottom surface of housing 202 and adhesive pad 204. CAM system 200 is configured to be worn on epidermis 104 of user 102 at a convenient location, such as the back of the upper arm, the abdomen, etc.

CAM system 200 may be battery powered, and, in certain embodiments, the battery may be replaced or recharged if necessary. SEM 220 is coupled to CAS 210, and includes electronic circuitry configured to acquire, process, store and transmit measured analyte data, as well as other information, to display devices 150 for presentation to user 102.

In certain embodiments, CAS 210 may be a single-analyte sensor that includes a percutaneous wire that has a proximal portion coupled to SEM 220 and a distal portion with several electrodes. A measurement (or working) electrode may be coated, covered, treated, embedded, etc., with one or more chemical molecules that react with a particular analyte, and a reference electrode may provide a reference electrical voltage. The measurement electrode may generate the analog sensor signal, which is conveyed along a conductor that extends from the measurement electrode to the proximal portion of the percutaneous wire that is coupled to SEM 220. After CAM system 200 has been applied to epidermis 104 of user 102, CAS 210 penetrates epidermis 104, and the distal portion extends into the dermis and/or subcutaneous tissue 106 under epidermis 104 (as depicted in FIG. 2B). Other configurations of CAS 210 may also be used, such as a multi-analyte sensor that includes multiple measurement electrodes, each generating an analog sensor signal that represents the concentration levels of a particular analyte.

In certain embodiments, CAS 210 may incorporate a thermocouple within, or alongside, the percutaneous wire to provide an analog temperature signal to SEM 220, which may be used to correct the analog sensor signal or the measured analyte data for temperature. In other embodiments, the thermocouple may be incorporated into SEM 220 above adhesive pad 204, or, alternatively, the thermocouple may contact epidermis 104 of user 102 through openings in adhesive pad 204.

In certain embodiments, SEM 220 includes, inter alia, processor (P) 222, memory (M) 224, transceiver or transmitter/receiver (T/R) 226, one or more antennae (A) 228 coupled to transceiver 226, analog signal processing circuitry, analog-to-digital (A/D) signal processing circuitry, digital signal processing circuitry, a power source for CAS 210 (such as a potentiostat), etc.

Processor 222 may be a general-purpose or application-specific microprocessor, an application-specific integrated circuit (ASIC), a field-programmable gate array (FPGA), etc., that executes instructions to perform control, computation, input/output, etc. functions for CAM system 200. Processor 222 may include a single integrated circuit, such as a micro-processing device, or multiple integrated circuit devices and/or circuit boards working in cooperation to accomplish the appropriate functionality. In certain embodiments, processor 222, memory 224, transmitter/receiver 226, the A/D signal processing circuitry, and the digital signal processing circuitry may be combined into a system-on-chip (SoC).

In operation, CAS 210 and adhesive pad 204 may be assembled to form an application assembly, where the application assembly is configured to be applied to the user's epidermis 104 so that CAS 210 is subcutaneously inserted as depicted. In such scenarios, SEM 220 may be attached to the assembly after application to the user's epidermis 104 via an attachment mechanism (not shown). Alternatively, SEM 220 may be incorporated as part of the application assembly, such that CAS 210, adhesive pad 204 and SEM 220 can all be applied at once to the user's epidermis 104. In one or more embodiments, this application assembly is applied to the user's epidermis 104 using a separate sensor applicator (not shown).

Unlike the fingersticks required by certain conventional analyte measurement techniques, for example, user-initiated application of CAM system 200 with a sensor applicator is nearly painless and does not require the withdrawal of blood. Moreover, the automatic sensor applicator generally enables the user to embed CAS 210 subcutaneously into the user's epidermis 104 without the assistance of a clinician or health care provider.

CAM system 200 may be removed by peeling adhesive pad 204 from the user's epidermis 104. It is to be appreciated that CAM system 200 and its various components are illustrated as one example form factor, and CAM system 200 and its components may have different form factors without departing from the spirit or scope of the described techniques.

Generally, processor 222 is configured to sample the analog sensor signal using the A/D signal processing circuitry at regular intervals (such as the sampling period), generate measured analyte data from the sampled analog sensor signal, and generate sensor data packages that include, inter alia, the measured analyte data. Processor 222 may store the measured analyte data in memory 224, and generate the sensor data packages at regular intervals (such as the transmission period) for transmission by T/R 226 to display device 150. Processor 222 may also add additional data to the sensor data packages, such as supplemental sensor information that includes a sensor identifier, a sensor status, temperatures that correspond to the measured analyte data, etc.

With respect to the supplemental sensor information, the sensor identifier represents information that uniquely identifies CAS 210 from other sensors, such as other sensors of other analyte monitoring devices, other sensors implanted previously or subsequently in the user's epidermis 104, and so on. By uniquely identifying CAS 210, the sensor identifier may also be used to identify other aspects about CAS 210, such as a manufacturing lot of CAS 210, packaging details of CAS 210, shipping details of CAS 210, and so on. In this way, various issues detected for sensors manufactured, packaged, and/or shipped in a similar manner as CAS 210 may be identified and used in different ways in order to calibrate the measured analyte data, to notify users of defective sensors, to notify manufacturing facilities of machining issues, and so forth.

The sensor status of the supplemental sensor information represents a state of CAS 210 at a given time, such as a state of the sensor at a same time one of the measured analyte data is produced. To this end, the sensor status may include an entry for each of the measured analyte data, such that there is a one-to-one relationship between the measured analyte data and statuses captured in the supplemental sensor information. For example, the sensor status may describe an operational state of CAS 210. In certain embodiments, processor 222 may identify one of a number of predetermined operational states for a given measurement. The identified operational state may be based on the communications from CAS 210 and/or characteristics of those communications.

In certain embodiments, a lookup table, stored in memory 224, may include the predetermined number of operational states and bases for selecting one state from another. For example, the predetermined states may include a “normal” operation state where the bases for selecting this state may include an analog sensor signal from CAS 210 that falls within thresholds indicative of normal operation, an analog temperature signal that is within a threshold of suitable temperatures to continue operation as expected, etc. The predetermined states may also include operational states that indicate that one or more characteristics of the analog sensor signal from CAS 210 are outside of normal activity and may result in potential errors in the measured analyte data, such as an analog sensor signal from CAS 210 that is outside a threshold of expected signal strength, an environmental temperature that is outside suitable temperatures to continue operation as expected, detecting that the user 102 has physically rolled onto CAM system 200, etc.

FIG. 3 illustrates input data 128 and metric data 130 for use by health management system 100, in accordance with embodiments of the present disclosure.

More particularly, FIG. 3 illustrates input data 128 on the left, display device 150 and network computing device 142 in the middle, and metric data 130 on the right. Generally, display device 150 stores and executes one or more related applications and presents GUI 160 to the user, while network computing device 142 stores and executes DSE 114 (including DAM 116), as well as other applications. As described above, in certain embodiments, a portion of DSE 114 may be stored on and executed by display device 150 (or CAM system 200), while the remaining portion of DSE 114 may be stored on and executed by network computing device 142. In other embodiments, DSE 114 may be stored on and executed by display device 150 (or CAM system 200).

In certain embodiments, metric data 130 includes various types of data, such as discrete numerical values, ranges, qualitative values (high/medium/low, stable/unstable, rate of change, points of inflection, etc.), etc. Display device 150 obtains input data 128 through one or more channels such as manual user input, sensors/monitors, other applications executing on display device 150, EMR systems, etc.). As mentioned above, in certain embodiments, DSE 114 (including DAM 116) may process input data 128 to generate metric data 130. For example, DSE 114 may process continuous analyte sensor data 129, such as measured glucose data provided by CAM system 200, to determine analyte features, such as glucose features 131.

In certain embodiments, starting with input data 128, food consumption information may include information about one or more of meals, snacks, and/or beverages, such as one or more of the size, content (milligrams (mg) of sodium, potassium, carbohydrate, fat, protein, etc.), sequence of consumption, and time of consumption. In certain embodiments, food consumption may be provided by a user through manual entry, by providing a photograph through an application that is configured to recognize food types and quantities, by scanning a bar code or menu, and/or interrogating an NFC/RFID tag. In various examples, meal size may be manually entered as one or more of calories, quantity (such as “three cookies”), menu items (such as “Royale with Cheese”), and/or food exchanges (such as 1 fruit, 1 dairy). In some examples, meal information may be received by the related application(s) executing on display device 150. In some examples, meal information may be provided via one or more other applications synchronized with the related application(s), such as one or more other mobile health applications executed by display device 150. In such examples, the synchronized applications may include, such as an electronic food diary application, photograph application, etc.

In certain embodiments, food consumption information entered by a user may relate to nutrients consumed by the user. Consumption may include any natural or designed food or beverage. Food consumption information entered by a user may also be related to analytes, including any of the other analytes described herein.

In certain embodiments, exercise information may also be provided. Exercise information may be any information surrounding activities, such as activities requiring physical exertion by the user. For example, exercise information may range from information related to low intensity (such as walking a few steps) and high intensity (such as five mile run) physical exertion. In certain embodiments, exercise information may be provided, for example, by an accelerometer sensor or a heart rate monitor on a wearable device such as a watch, fitness tracker, and/or patch.

In certain embodiments, user statistics, such as one or more of age, height, weight, BMI, body composition (such as % body fat), stature, build, or other information may also be provided as an input. In certain embodiments, user statistics may be provided through GUI 160, by interfacing with an electronic source such as an electronic medical record, from measurement devices, etc. In certain embodiments, the measurement devices include one or more of a wireless, such as a Bluetooth-enabled, weight scale or camera, which may, for example, communicate with display device 150 to provide user data.

In certain embodiments, treatment information may also be provided as an input. The treatment information may include information regarding different lifestyle habits, surgical procedures, and/or other non-invasive procedures recommended by the user's physician. For example, the user's physician may recommend a user increase/decrease their carbohydrate intake, exercise for a minimum of thirty minutes a day, or increase an insulin dosage or other medication to maintain, improve, and/or reduce hyper- and/or hypoglycemic episodes, etc. As another example, a healthcare professional may recommend that a user engage in at-home treatment and/or treatment at a clinic. The treatment information may also indicate a patient's adherence to the prescribed type, dosage, and/or timing of medications. For example, the treatment/medication information may indicate whether and when exactly and with what dosage/type the medication was taken.

In certain embodiments, measured analyte data may include glucose concentration levels measured by at least a glucose sensor (or multi-analyte sensor configured to measure at least glucose) that is a part of CAM system 200. Glucose baselines, glucose level rates of change, glucose trends, glucose variability, glucose clearance, glucose features, etc., may also be determined from the measured glucose data acquired by CAM system 200. Additionally, fasting blood glucose and HbA1c levels may be provided as metric data 130.

In certain embodiments, metric data 130 may include, inter alia, analyte features (such as glucose features 131) and related events 133. Glucose features 131 may include, inter alia, a glucose rapidly rising feature (also known as a glucose rapidly rising insight event), a glucose spike feature (also known as a glucose spike insight event, a glucose falling feature (also known as a glucose falling insight event), a glucose back-in-range feature (also known a glucose back-in-range insight event), a glucose time-in-range feature (also known as a glucose time-in-range insight event), etc. Related events 133 may include, inter alia, meal events, activity events, insulin dosing events, etc. Glucose insight events and related events are discussed in more detail below. Note that, alternatively or in addition to glucose features, lactate features, potassium features, creatinine features, or features associated with any other analyte described herein may be obtained and utilized according to the embodiments described herein

In certain embodiments, data may also be received from one or more non-analyte sensors 230. Data from non-analyte sensors 230 may include information related to a heart rate, heart rate variability (such as the variance in time between the beats of the heart), ECG data, a respiration rate, oxygen saturation, a blood pressure, or a body temperature (such as to detect illness, physical activity, etc.) of a user. In certain embodiments, electromagnetic sensors may also detect low-power radio frequency (RF) fields emitted from objects or tools touching or near the object, which may provide information about user activity or location.

In certain embodiments, data received from non-analyte sensors 230 may include data relating to a user's insulin delivery. In particular, data related to the user's insulin delivery may be received, via a wireless connection on a smart pen, via user input, and/or from an insulin pump. Insulin delivery information may include one or more of insulin manufacturer, insulin dosage, insulin formulation, insulin volume, basal vs bolus dose, intended pharmacokinetic profile (such as short-acting, long-acting), number of units of insulin delivered, time of delivery, etc. Other metrics, such as insulin action time or duration of insulin action, may also be received.

In certain embodiments, time may also be provided, such as time of day, UTC time or time from a real-time clock. Said real-time clock may be provided externally (synchronized to a server via a WiFi wireless connection) or may be embedded as an integrated circuit (RTC) within the wearable/sensor electronics. For example, measured analyte data may be timestamped to indicate a date and time when the analyte measurement was acquired by CAM system 200.

In certain embodiments, at least a portion of input data 128 may be acquired through GUI 160 of display device 150.

In certain embodiments, glucose concentration level rates of change may be determined from glucose measurement data. For example, a glucose concentration level rate of change refers to a rate that indicates how time-stamped glucose measurement data values change in relation to one or more other time-stamped glucose measurement data values. Glucose concentration level rates of change may be determined over one or more seconds, minutes, hours, days, etc.

In certain embodiments, a glucose trend may be determined based on glucose measurement data over a certain period of time. In certain embodiments, glucose trends may be determined based on glucose concentration level rates of change over certain periods of time.

In certain embodiments, glycemic variability may be determined from glucose measurement data. For example, glycemic variability refers to a standard deviation of glucose concentration levels over a period of time. Glycemic variability may be determined over one or more minutes, hours, days, etc.

In certain embodiments, a glucose clearance rate may be determined from glucose measurement data following consumption of a known, or estimated, amount of glucose or known nutrient resulting in production of glucose. Glucose clearance rates analyzed over time may be indicative of glucose homeostasis. The glucose clearance rate may be indicative of an effectiveness of a medication type, dosage, and/or frequency.

In certain embodiments, the glucose clearance rate may be determined by calculating a slope between an initial high glucose concentration level (such as a highest glucose concentration level during a period of 20-30 minutes after consumption of glucose) at t₀ and a subsequent low glucose concentration level at t₁. The low glucose concentration level (G_{L}) may be determined based on a user's initial high glucose concentration level (G_{H}) and a baseline glucose concentration level (G_{B}) before consumption of glucose. In certain embodiments, G_{L} can be a glucose concentration level between G_{H} and G_{B}, such as G_{L}=G_{B}+K*(G_{H}−G_{B})/2, where K can be a percentage representing by how much a user's glucose concentration level returned to user's baseline value. When K equals zero, low glucose concentration level equals baseline glucose value. When K equals 0.5, low glucose concentration level equals mean glucose concentration level between initial glucose concentration level and baseline glucose concentration level.

In certain embodiments, the glucose clearance rate may be determined over one or more periods of time after consumption of glucose, such as following an oral glucose tolerance test (OGTT). The glucose clearance rate may be calculated for each time period to represent dynamics of glucose clearance rate after consumption of glucose. These glucose clearance rates calculated over time may be time-stamped and stored in user's profile 118. Certain metrics may be derived from time-stamped glucose clearance rates, such as mean, median, standard deviation, percentile, etc.

In certain embodiments, health and sickness metrics may be determined, for example, based on one or more of user input (such as pregnancy information, known sickness or disease information, etc.), from physiologic sensors (such as temperature, etc.), activity sensors, etc. In certain embodiments, based on values of health and sickness metrics, a user's state may be defined as being one or more of healthy, ill, rested, or exhausted.

In certain embodiments, meal state metric may indicate state user is in with respect to food consumption. For example, meal state may indicate whether user is in one of a fasting state, pre-meal state, eating state, post-meal response state, or stable state. In certain embodiments, meal state may also indicate nourishment on board, such as meals, snacks, or beverages consumed, and may be determined, for example from food consumption information, time of meal information, and/or digestive rate information, which may be correlated to food type, quantity, and/or sequence (such as which food/beverage was eaten first).

In certain embodiments, meal habits metrics are based on content and timing of a user's meals. For example, if a meal habit metric is on a scale of 0 to 1, better/healthier meals user eats higher meal habit metric of user will be to 1, in an example. Also, the more the user's food consumption adheres to a certain time schedule or a recommended diet, closer their meal habit metric will be to 1, in an example.

In certain embodiments, an activity level metric may indicate user's level of activity. In certain embodiments, the activity level metric may be determined based on input from an activity sensor or other physiologic sensors, such as non-analyte sensors 230. In certain embodiments, activity level metric may be calculated by DAM 116 based on input data 128, such as one or more of exercise information, non-analyte sensor data (such as accelerometer data, etc.), time, user input, etc. In certain embodiments, the activity level metric may be expressed as a step rate of user. Activity level metrics may be time-stamped so that they may be correlated with one or more of the user's analyte levels at the same time.

In certain embodiments, body temperature metrics may be calculated by DAM 116 based on input data 128, and more specifically, non-analyte sensor data from a temperature sensor. In certain embodiments, heart rate metrics (such as heart rate and heart rate variability) may be calculated by DAM 116 based on input data 128, such as non-analyte sensor data from a heart rate sensor, etc. In certain embodiments, respiratory metrics (not shown) may be calculated by DAM 116 based on input data 128, such as non-analyte sensor data from a respiratory rate sensor, etc. In certain embodiments, blood pressure metrics (such as blood pressure levels and blood pressure trends) may be calculated by DAM 116 based on input data 128, such as non-analyte sensor data from blood pressure sensor, etc.

In certain embodiments, physiological metrics (such as analyte concentration levels, analyte concentration level rates of change, heart rate, blood pressure, etc.) associated with user may be stored as metric data 130 when a state or condition of user is confirmed. In certain embodiments, such physiological metrics may be analyzed over time to provide an indication of changes in state or condition of user.

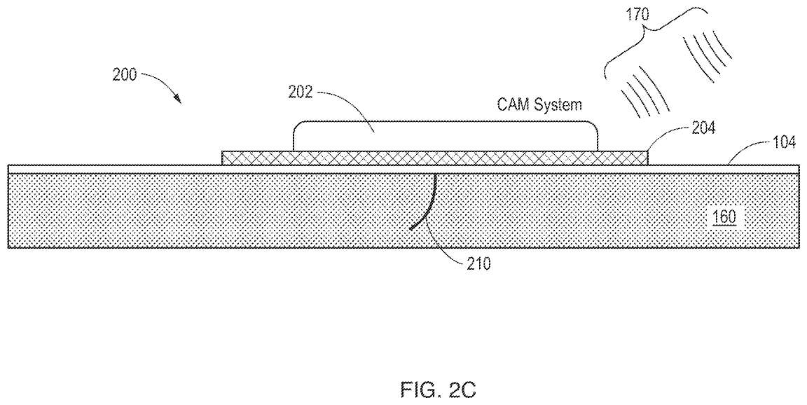

FIG. 4 depicts a block diagram of computing device 400, in accordance with embodiments of the present disclosure.

In certain embodiments, computing device 400 may be configured as display device 150. In these embodiments, computing device 400 may be coupled to network 180 via a wireless connection. Certain display devices 150, such as laptop computers, may include one or more I/O devices 435, such as a keyboard, a mouse, display 436, touch screen 437, etc. Other display devices 150, such as handheld health monitors, smartphones, smartwatches, tablet computers, etc., may include touch screen 437, which is a combination of an I/O device and a display. Other display devices 150, such as wearable health monitors, etc., may include one or more I/O devices 435 (such as buttons, a touchpad, etc.), and display 436 or touch screen 437. Generally, display devices 150 may be battery-powered, and the battery may be periodically recharged or replaced as needed.

In other embodiments, computing device 400 may be configured as network computing device 142, as well as the network computing device(s) of training system 140. In these embodiments, computing device 400 may be coupled to network 180 via a wired or wireless connection, and may include one or more optional I/O devices 435, such as a keyboard, a mouse, display 436, etc.

Computing device 400 includes interconnect (bus) 430 coupled to one or more processors 405, storage element or memory 410, one or more network interfaces 425, and one or more I/O interfaces 420, which may include a display interface (such as HDMI, etc.), a keyboard interface (such as USB, etc.), a local wireless communications interface (such as Bluetooth, BLE, RFID, NFC, etc.), a touch screen interface, etc. In certain embodiments, processor 405 may be a central processing unit (CPU), and computing device 400 may include one or more specialized processors, such as a graphics processing unit (GPU), a neural processing unit (NPU), etc. Generally, network interfaces 425 are coupled to network 180 using a wired or wireless connection(s), and I/O interfaces 420 are coupled to I/O device(s) 435, such as display 436, etc., using wired or wireless connections.

Bus 430 is a communication system that transfers data between processor 405, memory 410, network interfaces 425, and I/O interfaces 420. In certain embodiments, bus 430 transfers data between these components and one or more specialized processors, such as GPUs, NPUs, etc.

Processor 405 includes one or more general-purpose or application-specific microprocessors with one or more processing cores that execute instructions to perform various functions for computing device 400, such as control, computation, input/output, etc. Processor 405 may include a single integrated circuit, such as a micro-processing device, or multiple integrated circuit devices and/or circuit boards working in cooperation to accomplish the appropriate functionality. Additionally, processor 405 may execute software applications and software modules stored within memory 410, such as an operating system, DSE 114, etc. For example, DSE 114 may include rule-based models, machine learning models including LR models, ANNs, recurrent neural networks (RNNs), long short-term memory (LSTM) networks, convolutional neural networks (CNNs), etc., DAM 116, as well as other software modules.

Generally, memory 410 stores instructions for execution by processor 405 as well as data. Memory 410 may include a variety of non-transitory computer-readable medium that may be accessed by processor 405 as well as other components. In various embodiments, memory 410 may include volatile and nonvolatile medium, non-removable medium and/or removable medium. For example, memory 410 may include combinations of random access memory (RAM), dynamic RAM (DRAM), static RAM (SRAM), read only memory (ROM), flash memory, cache memory, and/or any other type of non-transitory computer-readable medium.

Memory 410 contains various components for retrieving, presenting, modifying, and storing user profile 118 as well as other data 412. For example, memory 410 stores software applications and modules that provide functionality when executed by processor 405, such as DSE 114, DAM 116, etc. The operating system provides operating system functionality for computing device 400. Data 412 may include data associated with the operating system, the software applications and modules, DSE 114, DAM 116, etc.

Network interfaces 425 are configured to transmit data to and from network 180 using one or more wired and/or wireless connections. As discussed above, network 180 may include one or more LANs, WLANs, LPWANs, WANs, cellular networks (such as 3G, 4G, LTE, 5G, 6G, etc.), the Internet, etc., employing various network topologies and protocols. For example, network 180 may also include various combinations of wired and/or wireless physical layers, such as, for example, copper wire or coaxial cable networks, fiber optic networks, WiFi networks, Bluetooth mesh networks, CDMA, FDMA and TDMA cellular networks, etc.

I/O interfaces 420 are configured to transmit and/or receive data from I/O devices 435. I/O interfaces 420 enable connectivity between processor 405, memory 410 and I/O device(s) 435 by encoding data to be sent from processor 405 or memory 410 to I/O devices 435, and decoding data received from I/O devices 435 for processor 405 or memory 410. Generally, data may be sent over wired and/or wireless connections. For example, I/O interfaces 420 may include one or more wired communications interfaces, such as USB, Ethernet, etc., and/or one or more wireless communications interfaces, coupled to one or more antennas, such as WiFi, Bluetooth, cellular, etc. Importantly, CAM system 200 may communicate with I/O interfaces 420 via Bluetooth, BLE, RFID, NFC, etc.

Generally, I/O devices 435 provide data to and from computing device 400. As discussed above, I/O devices 435 are operably connected to computing device 400 using a wired and/or wireless connection. I/O devices 435 may include a local processor coupled to a communication interface that is configured to communicate with computing device 400 using the wired and/or wireless connection. For example, I/O devices 435 may include display 436, touch screen 437, a keyboard, a mouse, a touch pad, etc.

Generally, DSE 114 may generate insight notifications based on metric data 130, which includes measured analyte data provided by the CAM sensor device worn by the user (such as measured glucose data provided by a CGM sensor device). In certain embodiments, DSE 114 may be stored on and executed by display device 150, which may also store at least a relevant portion of metric data 130. Accordingly, display device 150 may generate and present the insight notifications to the user. In other embodiments, DSE 114 may be stored on and executed by network computing device 142, which may generate the insight notifications based on metric data 130, and then transmit the insight notifications to display device 150 for presentation to the user.

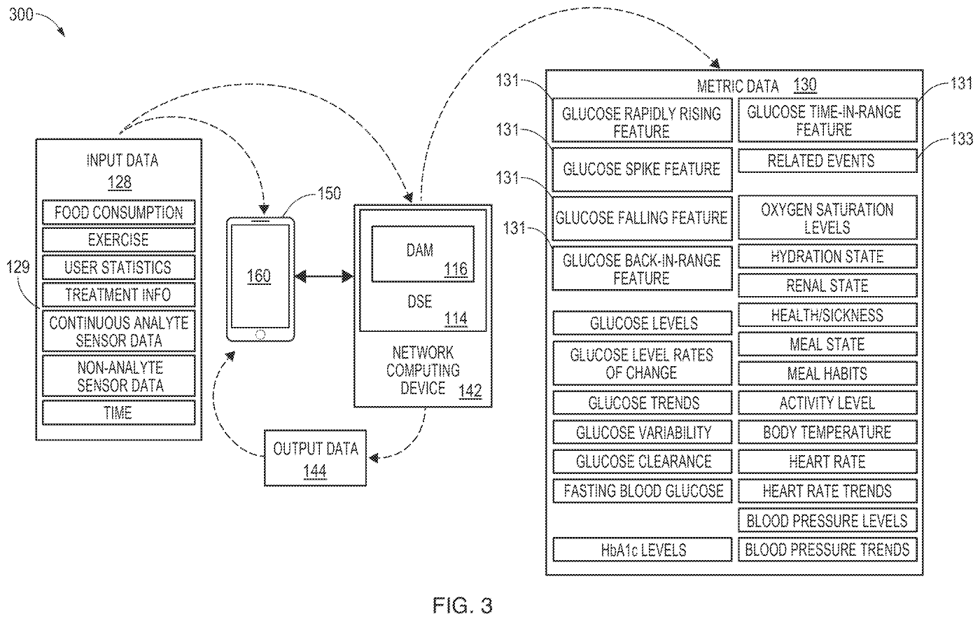

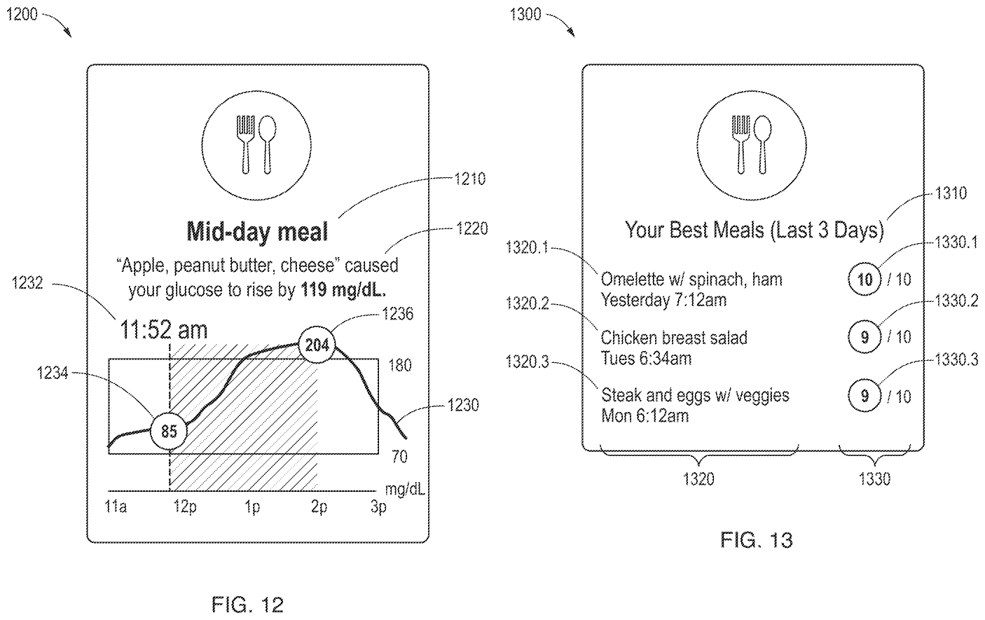

The insight notifications help the user determine why analyte level fluctuations may be contemporaneously occurring, such as how the user's recent meals, activities, etc., may have impacted the user's measured glucose concentration levels, etc. FIGS. 5A to 11 provide examples of insight notifications for an analyte rapidly rising insight, an analyte spike insight, an analyte falling insight, an analyte back-in-range insight, and an analyte time-in-range insight, while FIGS. 12 to 14B provide meal and other related event notifications.

FIG. 5A depicts analyte rapidly rising insight notification 500, in accordance with embodiments of the present disclosure.

The analyte rapidly rising insight notification (or rapid rise notification) 500 may be generated and presented in real time or near real time when an analyte rapidly rising insight event (or rapid rise event) is detected by DSE 114. Generally, the analyte concentration levels are “rapidly rising” when the analyte concentration levels are increasing at a rapid pace over a short period of time. For purposes of illustration, the analyte is glucose.

In certain embodiments, the analyte rapid rise notification 500 may include an image 510 of the rising analyte concentration level, and alphanumeric text 520 indicating, inter alia, the time that the analyte concentration level started rising, as depicted in FIG. 5A. Generally, the analyte rapid rise event reflects a rapid rise of analyte concentration level that is approaching (or has exceeded) the upper limit of the normal (target) range for the user.

In certain embodiments, the analyte rapid rise notification 500 may also include suggested activities that may be undertaken to slow, stop, and reverse the rapidly rising analyte concentration level, such as drink water, go for a walk, take a deep breath (waiting it out), etc. The analyte rapid rise notification 500 advantageously provides a type 2 user the opportunity to modify their behavior while the hyperglycemic event is occurring to very quickly address and reduce hyperglycemia.

FIG. 5B depicts analyte rapidly rising insight notification 502, in accordance with embodiments of the present disclosure. The analyte rapid rise notification 502 may include an image 530 of the rising analyte concentration level over time (such as a graph, etc.), and alphanumeric text block 540 indicating that the analyte concentration level is rising.

FIG. 5C depicts analyte rapidly rising insight notification 504, in accordance with embodiments of the present disclosure. The analyte rapid rise notification 504 may include an image 540 of the rising analyte concentration level (such as a graph, etc.), and alphanumeric text 540 indicating that the analyte concentration level is rising. Other relevant information may also be presented.

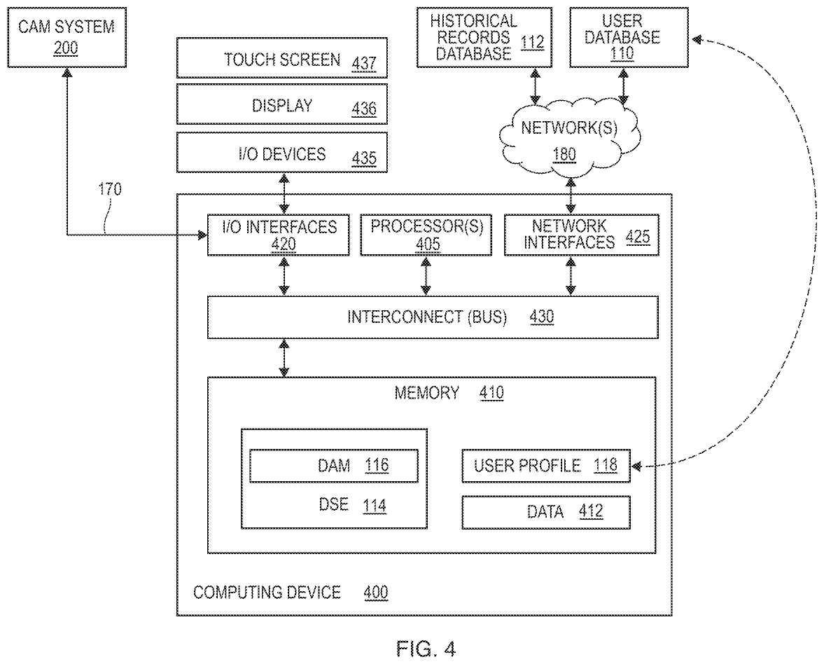

FIG. 6 depicts analyte spike insight notification 600, in accordance with embodiments of the present disclosure.

The analyte spike insight notification 600 may be generated and presented periodically (such as daily, etc.), and summarizes the characteristics of each analyte spike insight event detected over a predetermined time period (such as 24 hours, etc.). For purposes of illustration, the analyte is glucose.

In certain embodiments, the analyte spike insight notification 600 may include an image 610 of the analyte concentration level spike, and alphanumeric text 620 indicating, inter alia, the amount of analyte concentration level increase during the analyte spike insight event, as well as the start and end times of the analyte spike insight event, as depicted in FIG. 6. The analyte spike insight notification 600 may also present the analyte concentration levels at the start and end of the analyte spike insight event, the highest peak analyte concentration level, the duration of the spike, etc. Generally, the analyte spike insight event reflects a rapid rise and then peak of analyte concentration level above the upper limit of the normal (target) range for that user, followed by a fall of analyte concentration level back to the normal (target) range for that user. One or more target ranges may be customized for each user.

DSE 114 may also compare each analyte spike insight event to the user's normal analyte concentration level patterns, which may help the user understand and pinpoint the behavior that may have caused the analyte concentration levels to spike, i.e., rise, peak, and fall. In certain embodiments, display device 150 may present each analyte spike insight event to the user within an interactive analyte insight event log over a predetermined time period, such as daytime, nighttime, etc. When the user selects one of the analyte spike insight events in the log, the detail related to the selected analyte spike insight event may be presented in a pop up window, etc. DSE 114 may also apply a spike rating to each analyte spike insight event for comparison purposes, such as a subjective label (small, medium, large), an objective score (numeric value), etc.

Conversely, DSE 114 may also generate an analyte dip insight notification in real time or near real time when an analyte dip insight event is detected. The analyte dip insight notification advantageously provides the opportunity for a user to modify their behavior while the analyte dip insight event is occurring to very quickly address hypoglycemia. Generally, the analyte dip insight event reflects a rapid fall and then a dip of analyte concentration level below the lower limit of the normal (target) range for the user, followed by a rise of analyte concentration level back to the normal (target) range for the user.

Advantageously, the interactive analyte insight event log may help users recall what happened during each analyte insight event, and may include GUI control elements (such as a record button, etc.) to allow the user to record (store) an event that may be related to the analyte insight event (such as a meal, an activity, etc.), a free form note, etc. The related event may be stored in a related event log with an event type, a date, and a time. While the user may review their measured analyte data for analyte spikes and dips using a landscape GUI display mode and data scrubbing feature, the interactive analyte insight event log advantageously presents the measured analyte data in an easily-accessible format that encourages the user to store (create) related events in the related event log.

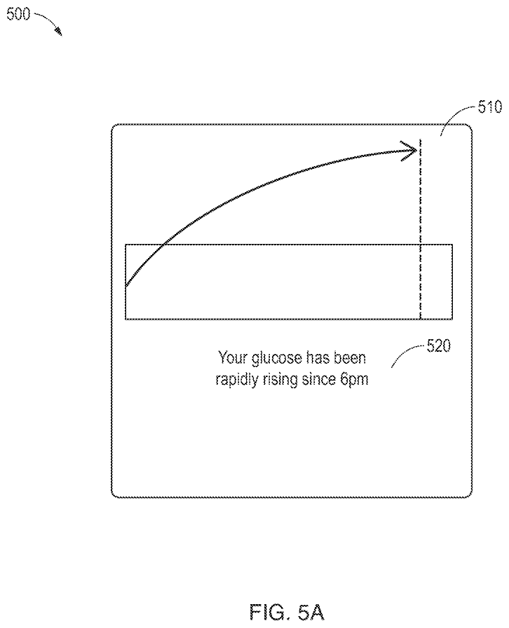

FIG. 7 depicts analyte spike insight notification 700, in accordance with embodiments of the present disclosure.

The analyte spike insight notification 700 may be generated in real time or near real time when the start of an analyte spike insight event is detected by DSE 114. Typically, the analyte spike insight notification 700 may be generated and presented during daytime hours (such as 6 am to 6 pm), during the user's waking hours determined from data from a 3^{rd} party system that measures sleep (such as a CPAP, a wearable health monitoring device, etc.), etc. Additionally, the analyte spike insight notification 700 may be generated and presented along with the analyte spike insight notification 600 to store an event that may be related to the analyte spike insight event.

In certain embodiments, the analyte spike insight notification 700 may include alphanumeric text 620 indicating, inter alia, the start time of the analyte spike insight event, and several GUI control elements 730, as depicted in FIG. 7. GUI control elements 730 may access additional functionality related to analyte spike insight notification 700, such as creating an entry in the related event log to document a meal event (Meal button 731), an activity event (Activity button 732), another event (Other button 733) such as a medicament (such as insulin) dosing event, etc., as well as a GUI control element to dismiss the analyte spike insight notification 700 (Not Now button 734).

In certain embodiments, in response to the selection of the Not Now button 734, display device 150 may create an entry in the related event log with a date, a time, and an event type of “non-logged event.” The “non-logged event” event type creates a placeholder for additional related event data that may be used to increase the robustness of the glucose insight event detection process.

In other embodiments, display device 150 may be wirelessly connected to a wearable health monitoring device (such as a Fitbit, a smartwatch, etc.) that monitors and records various user events, such as meals, activities (such as exercise, etc.), health events (such as a rise in stress, etc.), etc. Advantageously, display device 150 may request these data from the wearable health monitoring device, compare the date and time of the user events to the analyte insight event log, and then prompt the user to confirm the entry of the user event into the related event log, such as “We noticed that you ran at 12 pm and we made a note of it, is this correct?”.

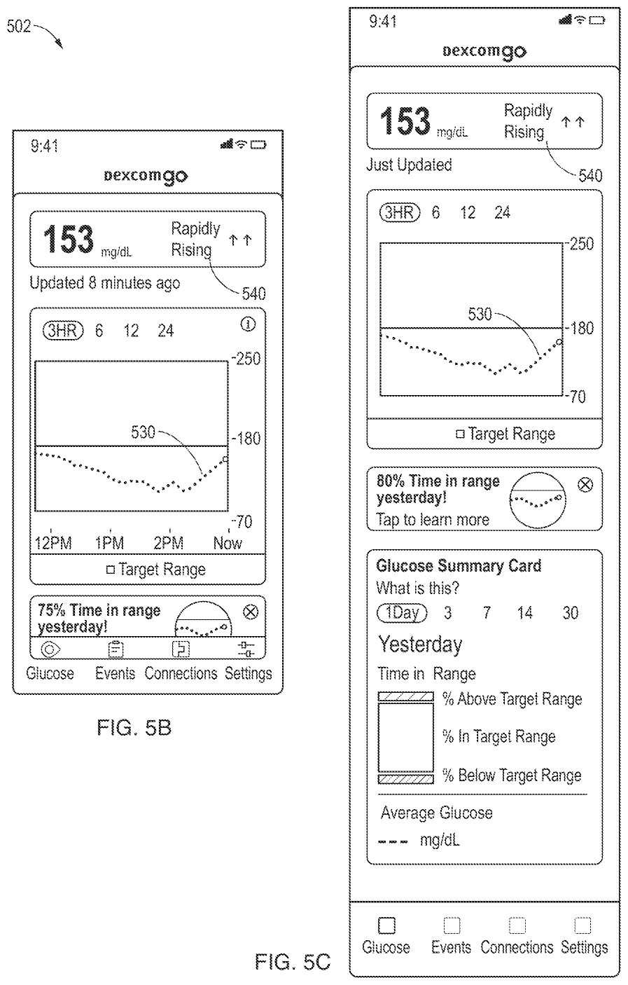

FIG. 8 depicts analyte falling insight notification 800, in accordance with embodiments of the present disclosure.

The analyte falling insight notification 800 may be generated in real time or near real time when an analyte falling insight event is detected by DSE 114, such as when the analyte concentration level is falling (coming down) after the peak of an analyte spike insight event, etc. For purposes of illustration, the analyte is glucose.

In certain embodiments, the analyte falling insight notification 800 may include an image 810 of the falling analyte concentration level, and alphanumeric text 820 indicating, inter alia, that the analyte concentration level is coming down from a spike, as depicted in FIG. 8. The analyte falling insight notification advantageously provides reassurance to the user that their analyte concentration level is falling after an analyte spike insight event has occurred.

Conversely, DSE 114 may also generate an analyte rising insight notification in real time or near real time when an analyte rising insight event is detected, such as when the analyte concentration level is rising (coming up) after the nadir of an analyte dip insight event, etc. The analyte rising insight notification advantageously provides reassurance to the user that their analyte concentration level is rising after an analyte dip insight event has occurred.

Accordingly, the analyte falling insight notification 800 and the analyte rising insight notification advantageously provide reassurance to the diabetic user after their glucose concentration level has spiked or dipped, and may particularly reduce the anxiety, fear, or even panic of diabetes type 2 users as they check their glucose concentration levels every few minutes waiting for it to stabilize.

For example, if the glucose concentration level falls back into range (for that user) within 2 hours, information may be presented to the user to reassure them that a spike after a meal that comes back into the range is an acceptable response. If the fall back into range is longer that the recommended time (such as greater than 2 hours), display device 150 may provide advice to the user on goals (such as for the glucose concentration levels to be back in range after 2 hours, etc.), and may provide one or more suggestions for future actions for a more optimal response (such as eat less, go for a walk, get a drink, etc.).

As noted above, embodiments of the present disclosure may be applied to other diseases and their associated analytes. In other words, an analyte falling insight notification may be generated and presented in real time or near real time when an analyte falling insight event is detected by DSE 114, and an analyte rising insight notification may be generated and presented in real time or near real time when an analyte rising insight event is detected by DSE 114.

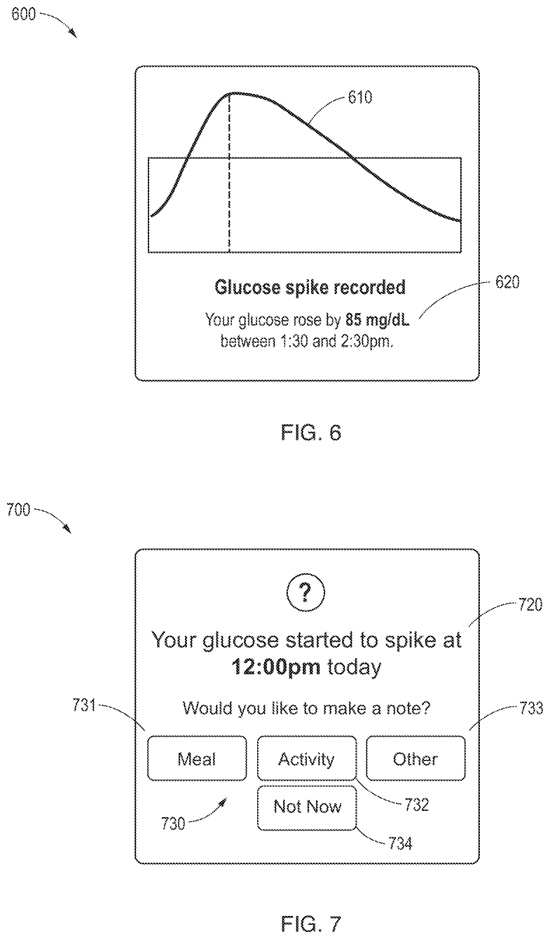

FIG. 9 depicts analyte back-in-range insight notification 900, in accordance with embodiments of the present disclosure.

The analyte back-in-range insight notification 900 may be generated and presented in real time or near real time after an analyte falling (or rising) insight event was detected by DSE 114. For purposes of illustration, the analyte is glucose.

In certain embodiments, the analyte back-in-range insight notification 900 may include an image 910 of the falling (or rising) analyte concentration level as it approaches the normal (target) analyte range for the user, and alphanumeric text 920 indicating that the analyte concentration level is back in range (i.e., less than an upper range threshold level and greater than a lower range threshold level), as depicted in FIG. 9. The analyte back-in-range insight notification advantageously provides reassurance to the diabetic user that their glucose concentration level has reached their normal (target) range, and may particularly reduce the anxiety, fear, or even panic of diabetes type 2 users as they check their glucose concentration levels every few minutes waiting for it to stabilize.

For example, if the glucose concentration level is back into range (for that user) after 2 hours, information may be presented to the user to reassure them that a spike after a meal that comes back into the range is an acceptable response. If the glucose concentration level is not back into range by the recommended time (such as 2 hours), display device 150 may provide advice to the user on goals (such as for the glucose concentration levels to be back into range after 2 hours, etc.), and may provide one or more suggestions for future actions for a more optimal response (such as eat less, go for a walk, get a drink, etc.).

As noted above, embodiments of the present disclosure may be applied to other diseases and their associated analytes. In other words, an analyte back-in-range insight notification may be generated and presented in real time or near real time when an analyte falling (or rising) insight event is detected by DSE 114.

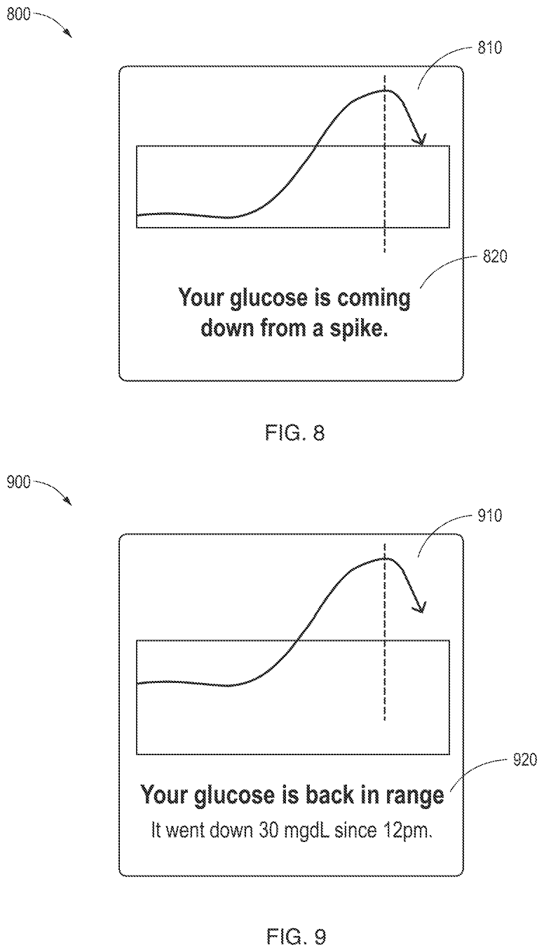

FIG. 10 depicts analyte time-in-range insight notification 1000, in accordance with embodiments of the present disclosure.

The analyte time-in-range insight notification 1000, 1100 may be generated periodically (such as at various times during the day, once a day, etc.), and summarizes the analyte time-in-range insight event detected by DSE 114 over a predetermined time period (such as from midnight to the current time, the previous day, each of the previous 3 days, each of the previous 5 days, etc.). For purposes of illustration, the analyte is glucose.

In certain embodiments, the analyte time-in-range insight notification 1000 may include a number 1010 representing the percentage of time that the user's analyte concentration levels have remained within the normal (target) range during the current day (i.e., from midnight to the current time), and alphanumeric text 1020 presenting encouragement and advice with respect to the analyte time-in-range insight event, as depicted in FIG. 10.

In other words, the analyte time-in-range insight notification 1000 summarizes the user's current analyte time-in-range progress so far for the current day. DSE 114 periodically updates the analyte time-in-range insight event throughout the current day, and the analyte time-in-range insight notification 1000 may be accessed by the user at any time (via a GUI control element, a pull-down menu, etc.) in order to track progress towards the normal (target) time-in-range (such as 70%). In certain embodiments, the normal (target) time-in-range may be customizable by the user.

If the current analyte time-in-range is above the normal (target) time-in-range, alphanumeric text 1020 may include a celebratory message. Conversely, if the current analyte time-in-range is below the normal (target) time-in-range, alphanumeric text 1020 may include one or more suggestions for improving the user's analyte time-in-range performance. Advantageously, alphanumeric text 1020 may also include contextual messages that depend on the current time within the current day (24-hour period). For example, if the current time is 5 pm, the contextual message may include suggested dinner choices that may help the diabetic user achieve or maintain the normal (target) time-in-range. If the current time is after 1 pm (with a noon meal stored in the related event log), the contextual message may include suggested exercise, such as taking a walk.

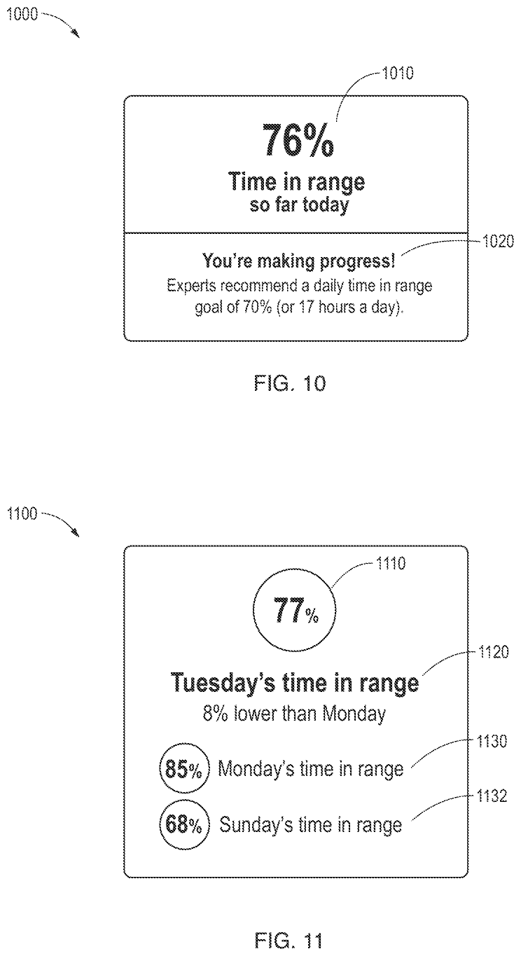

FIG. 11 depicts analyte time-in-range insight notification 1100, in accordance with embodiments of the present disclosure.

In certain embodiments, the analyte time-in-range insight notification 1100 may include a number 1110 representing the percentage of time that the user's analyte concentration levels remained within the normal (target) range the previous day, and alphanumeric text 1120 presenting a comparison of the analyte time-in-range insight events for each day of the previous two days, as depicted in FIG. 11. The comparison of the analyte time-in-range insight events for the previous two days may include a “+/−%” time-in-range (TIR) delta value from the preceding day (such as “Tuesday's time in range [was] 8% lower than Monday”). The analyte time-in-range insight notification 1100 may also include alphanumeric text 1130 presenting the analyte time-in-range insight event for two days in the past, and alphanumeric text 1132 presenting the analyte time-in-range insight event for 3 days in the past. The analyte time-in-range insight notification 1100 may be presented to the user at a fixed time each day, such as the morning, etc.

In other words, the analyte time-in-range insight notification 1100 summarizes the user's daily analyte time-in-range insight events for the past 3 days. In certain embodiments, the analyte time-in-range insight notification 1100 may summarize the user's daily analyte time-in-range insight events for the past 5 days, 7 days, 10 days, 15 days, 30 days, etc. In certain embodiments, color coding may be used to compare the daily time-in-range to the recommended (target) time-in-range for the user, as well as to distinguish between a customizable (target) time-in-range and a system default setting.

If one or more of the daily analyte time-in-range for the past 3 days (or the past 5 days, 7 days, etc.) is above the normal (target) time-in-range, the analyte time-in-range insight notification 1100 may also include a celebratory message. Conversely, if one or more of the daily analyte time-in-range for the past 3 days (or the past 5 days, 7 days, etc.) is below the normal (target) time-in-range or decreased from the previous day or two, the analyte time-in-range insight notification 1100 may also include one or more contextual messages for improving the user's daily analyte time-in-range performance.

Advantageously, the contextual messages may be based on analyte measurement data for the past 3 days (or the past 5 days, 7 days, etc.) as well as events stored within the related events log. For example, if the diabetic user recorded a late dinner in the related events log and subsequently experienced a large glucose spike insight event which caused the daily glucose time-in-range to decrease from the previous day or days, the contextual messages may include eating an earlier dinner with less carbohydrates, go for a walk after dinner, etc.

FIG. 12 depicts meal related event notification 1200, in accordance with embodiments of the present disclosure.

In certain embodiments, the meal related event notification 1200 may include a meal type 1210 (such as “mid-day meal,” “evening meal,” etc.), and alphanumeric text 1220 describing the content of the meal (such as “Apple, peanut butter, cheese”), as depicted in FIG. 12. In certain embodiments, meal type 1210 may be a predefined meal description that is selected from a list provided to the user when the meal data is entered into the related event log, such as “breakfast,” “lunch,” “dinner,” “snack,” etc. For purposes of illustration, the analyte is glucose.

The meal data may be entered by the user and stored in the related event log, meal data received from a 3^{rd} party system and then stored in the related event log, etc. The meal data stored in the related event log may include not only event type (i.e., meal), date, and start time, but also meal type 1210, meal content, post-prandial time period, analyte concentration level at the meal start time, analyte concentration level at the end of the post-prandial time period, as well as other data.

The meal related event notification 1200 may present analyte concentration level information to the user for a period of time subsequent to the meal (i.e., the post-prandial time period, such as 1 hour, 2 hours, etc.), such as a graph of analyte concentration levels 1230, a meal start time 1232 (depicted as 11:52 am, etc.), a analyte concentration level 1234 at the meal start time (depicted as 85 mg/dL), a analyte concentration level 836 at 2 hours past the meal start time (depicted as 204 mg/dL, etc.), etc., as depicted in FIG. 12. Additionally, the difference in analyte concentration levels between the meal start time and 2 hours past the meal start time may be included in alphanumeric text 1220 (such as “[this meal] caused your glucose to rise by 119 mg/dL”, etc.).

In other words, the meal related event notification 1200 not only provides information about the meal but also provides analyte concentration levels at the start of the meal, during the meal, 1 hour past the meal start time, 2 hours past the meal start time, etc. Because 1-hour and 2-hour post-prandial analyte concentration levels may be used by physicians as indicators of metabolic health, monitoring glucose concentration levels after meals advantageously provides an insight into how the user's body responds to the meals. In certain embodiments, the post-prandial time period may be customizable by the user.

DSE 114 may also determine a meal rating based on the characteristics of the analyte concentration levels between the meal start time and the end of the post-prandial time period, such as a subjective label (small, medium, large), an objective score (a numeric value, such as 1 to 10, etc.), etc. The characteristics may include the amount of the rise in the analyte concentration level, the peak analyte concentration level, the duration of the rise in analyte concentration level, etc.

For example, DSE 114 may evaluate the duration of the rise in glucose concentration level by comparing the glucose concentration level at a fixed interval post prandial time (such as 2 hours) to the upper limit of the recommended range to determine whether the user came back into the recommended range (or not). If the time needed for the glucose concentration level to come back down was longer for certain meals, then DSE 114 may assign a lower score to those meals. Similarly, DSE 114 may evaluate the rate of the rise in glucose concentration level, and may assign a lower score to meals when the rate of the rise was rapid. In other words, the glucose concentration level rose quickly after the meal. DSE 114 may evaluate the duration of the rise and the rate of the rise in combination to determined the score.

The meal related event notification 1200 may include additional alphanumeric text that includes the meal rating to help the user determine how the meal may have impacted the user's analyte concentration levels. In certain embodiments, a meal icon may be displayed in the analyte concentration level data (such as a trend graph, etc.), and the meal related event notification 1200 may be presented to the user when the meal icon is selected.

DSE 114 may also compare meal data (such as post-prandial glucose concentration levels, meal ratings, etc.) from different meals over a particular time period to determine which meals had the greatest impact (positive or negative) on the diabetic user's glucose concentration levels, and to provide insights into how the user's body responds to different meals.

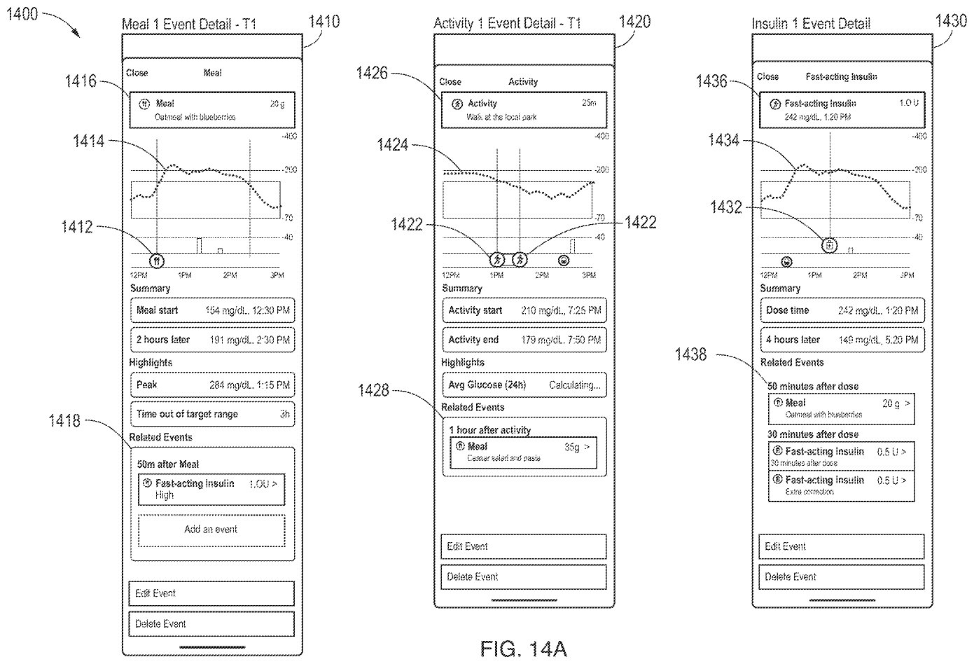

FIG. 13 depicts meal related event notification 1300, in accordance with embodiments of the present disclosure.

In certain embodiments, the meal related event notification 1300 may include alphanumeric text 1310 describing a meal comparison time period (such as 1 day, 2 days, 3 days, 5 days, 7 days, etc.), alphanumeric text 1320 presenting a description of a number of meals over the meal comparison time period (such as 3 meals, 6 meals, all the lunches, all the dinners, all the meals, etc.), and alphanumeric text 1330 representing the meal rating for each meal, as depicted in FIG. 13. The meal related event notification 1300 may help the user identify which meals maintained analyte concentration levels within the normal (target) range (positive impact), as well as which meals should be avoided or limited due to adverse analyte concentration levels effects, such as analyte spikes (negative impact).

For example, for a 3 day comparison period, alphanumeric text 1320.1 may describe the contents and start time of the highest rated meal and alphanumeric text 1330.1 may preset the meal rating for this meal, alphanumeric text 1320.2 may describe the contents and start time of the next highest rated meal and alphanumeric text 1330.2 may preset the meal rating for this meal, alphanumeric text 1320.3 may describe the contents and start time of the next highest rated meal and alphanumeric text 1330.3 may preset the meal rating for this meal, and so on. The meal related event notification 1300 may be presented to the user at a fixed time each day, such as the morning, at noon, in the afternoon, etc.

For another example, for a 3 day comparison period, alphanumeric text 1320.1 may describe the contents and start time of the lowest rated meal and alphanumeric text 1330.1 may preset the meal rating for this meal, alphanumeric text 1320.2 may describe the contents and start time of the next lowest rated meal and alphanumeric text 1330.2 may preset the meal rating for this meal, alphanumeric text 1320.3 may describe the contents and start time of the next lowest rated meal and alphanumeric text 1330.3 may preset the meal rating for this meal, and so on.

Display device 150 may display meals, activities, and medicament dosing events along with measured analyte data (such as a trend graph) to better understand how these events impact the user's measured analyte patterns. For purposes of illustration, the analyte is glucose, the disease is diabetes, and the medicament is insulin.

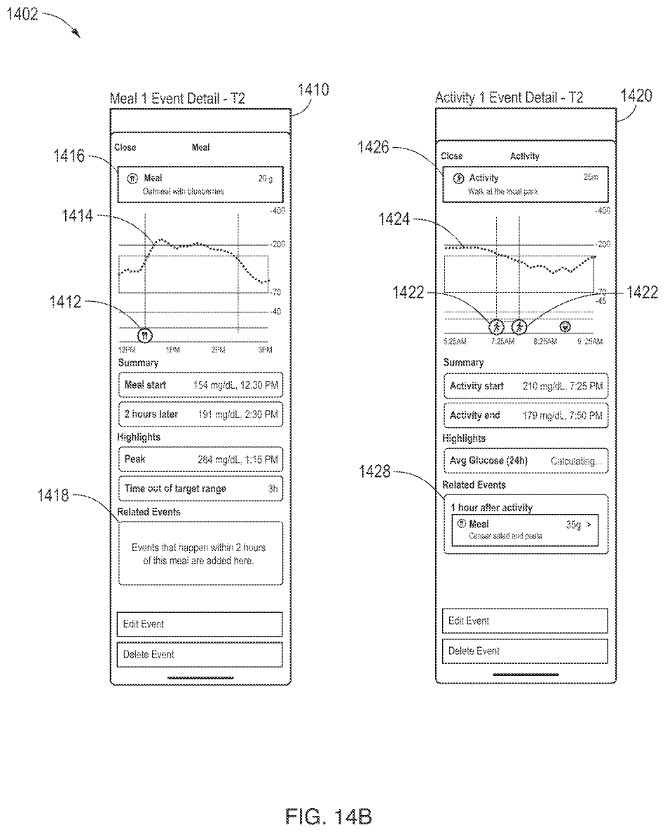

FIG. 14A depicts related event notifications 1400, in accordance with embodiments of the present disclosure.

In certain embodiments, related event notifications 1400 may include certain related events for user's with diabetes treated with insulin, such as meal event notification 1410, activity event notification 1420, and insulin dosing event notification 1430.

In certain embodiments, meal event notification 1410 may include, inter alia, meal icon 1412 displayed at the meal start time, glucose concentration level 1414 over time (depicted as a trend graph), alphanumeric text 1416 describing the content of the meal (such as “20 g Oatmeal with blueberries”), and related event information 1418 (such as an insulin dosing event 50 min. after the meal).

In certain embodiments, activity event notification 1420 may include, inter alia, activity icons 1422 displayed at the activity start time and the activity end time, glucose concentration level 1424 over time (depicted as a trend graph), alphanumeric text 1426 describing the type of activity (such as “25 m Walk at the local park”), and related event information 1428 (such as a meal event 1 hour after the activity).

In certain embodiments, insulin dosing event notification 1430 may include, inter alia, insulin dosing icon 1432 displayed at the insulin dose time, glucose concentration level 1434 over time (depicted as a trend graph), alphanumeric text 1436 describing the characteristics of the insulin dose (such as “Fast-acting Insulin 1.0 U 242 mg/dL, 1:20 pm”), and related event information 1438 (such as a meal event 50 min. after the dose, two insulin dosing events 30 min. after the dose).

FIG. 14B depicts related event notifications 1402, in accordance with embodiments of the present disclosure.

In certain embodiments, related event notifications 1402 may include certain related events for user's with type 2 diabetes, such as meal event notification 1410, and activity event notification 1420.

In certain embodiments, meal event notification 1410 may include, inter alia, meal icon 1412 displayed at the meal start time, glucose concentration level 1414 over time (depicted as a trend graph), alphanumeric text 1416 describing the content of the meal (such as “20 g Oatmeal with blueberries”), and related event information 1418.

In certain embodiments, activity event notification 1420 may include, inter alia, activity icons 1422 displayed at the activity start time and the activity end time, glucose concentration level 1424 over time (depicted as a trend graph), alphanumeric text 1426 describing the type of activity (such as “25 m Walk at the local park”), and related event information 1418 (such as a meal event 1 our after the activity).

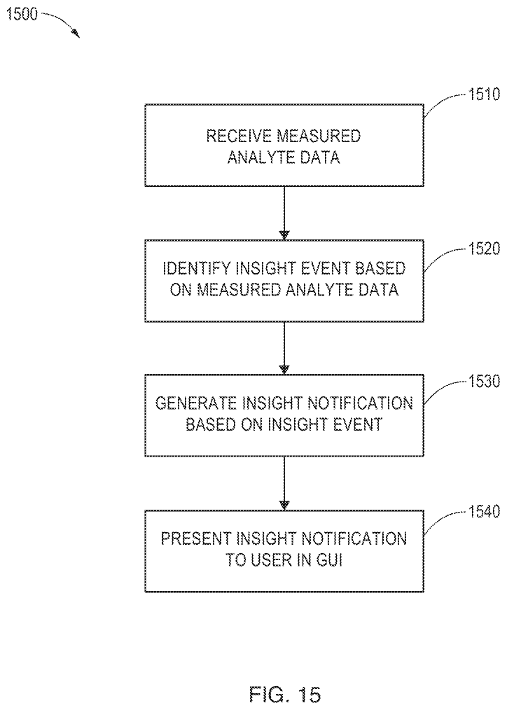

FIG. 15 depicts process flow diagram 1500 for providing insight notifications on a display device, in accordance with embodiments of the present disclosure.

As described above, embodiments of the present disclosure advantageously provide a display device that not only presents measured analyte data to the user, but also generates and presents contemporaneous insight notifications to the user based on the measured analyte data (such as measured glucose data, etc.).

At block 1510, measured analyte data are received from a CAM sensor device worn by a user. For example, measured glucose data may be received from a CGM sensor device.

At block 1520, an insight event is identified based on the measured analyte data within a predetermined time period. For example, DSE 114 may analyze the user's measured analyte data over the past 5 minutes, 10 minutes, 30 minutes, the past hour, the past 3 hours, etc., in order to identify insight events. In another example, DSE 114 may also analyze the user's measured analyte data over a somewhat longer predetermined time period, such as the current day, the past day, the past several days, eth past 5 days, the past 7 days, etc. For example, DSE 114 may identify the insight event based on measured glucose data within the predetermined time period.

Exemplary insight events may include an analyte rapidly rising insight event, an analyte falling insight event, an analyte falling (or rising) insight event, an analyte time-in-range insight event, etc. Embodiments of the present disclosure are not limited to these examples.

At block 1530, an insight notification is generated based on the insight event. The insight notification helps the user determine why analyte level fluctuations may be contemporaneously occurring, such as how the user's recent meals, activities, etc., may have impacted the user's measured analyte concentration levels. For example, the insight notification may help the user determine how the user's recent meals, activities, etc., may have impacted the user's measured glucose concentration levels.

At block 1540, the insight notification is presented to the user in a GUI.

### Example Clauses

Implementation examples are described in the following numbered clauses:

Clause 1: A method for providing insight notifications on a display device, the method comprising receiving measured analyte data from a continuous analyte monitoring sensor device worn by a user; identifying an insight event based on the measured analyte data within a predetermined time period, wherein the predetermined time period is between 5 minutes and 3 days; generating an insight notification based on the insight event, the insight event including one of an analyte rapidly rising insight event, an analyte spike insight event, an analyte falling insight event, an analyte back-in-range insight event, and an analyte time-in-range insight event; and presenting the insight notification to the user in a graphical user interface (GUI).

Clause 2: The method according to Clause 1, wherein the insight event is rapidly rising insight event; and the insight notification is an analyte rapidly rising insight notification that includes a graph of analyte concentration level over time, and an alphanumeric text block including an amount of analyte concentration level increase during the analyte spike insight event, and a start time and an end time of the analyte spike insight event.

Clause 3: The method according to Clause 1, wherein the insight event is the analyte spike insight event; and the insight notification is an analyte spike insight notification that includes a graph of analyte concentration level over time, and an alphanumeric text block indicating that the analyte concentration level is rising.

Clause 4: The method according to Clause 1, wherein the insight event is the analyte back-in-range insight event; and the insight notification is an analyte back-in-range insight notification that includes a graph of analyte concentration level over time, and an alphanumeric text block indicating that the analyte concentration level is less than an upper range threshold level and greater than a lower range threshold level.

Clause 5: The method according to Clauses 1, 2, 3, or 4, wherein the method further comprises determining an occurrence of a related event within the predetermined time period, the related event comprising a meal, an activity, or a medicament dosing.

Clause 6: The method according to Clause 5, wherein determining the occurrence of the related event is based on related event data received from the user, the related event data including an event type, a date, and a time.

Clause 7: The method according to Clause 6, further comprising generating a related event notification based on the related event, the related event notification including a graph of analyte concentration levels over time, an icon displayed at a related event start time, and an alphanumeric text block describing the related event; and presenting the related event notification to the user in the GUI, when the related event is a meal, the icon is a meal icon and the alphanumeric text block describes a content of the meal; when the related event is an activity, the icon is an activity icon and the alphanumeric text block describes the activity; and when the related event is a medicament dosing, the icon is a medicament dosing icon and the alphanumeric text block describes the medicament dosing.

Clause 8: The method according to any of Clauses 1 to 7, wherein the predetermined time period is between 5 minutes and 30 minutes, 30 minutes and 3 hours, or 3 hours and 3 days; the measured analyte data are measured glucose data; and the insight notification comprises one or more images, numbers, alphanumeric text, or graphical control elements.

Clause 9: A display device, comprising a wireless transceiver configured to receive measured analyte data from a continuous analyte monitoring sensor device worn by a user; a memory comprising executable instructions; and a processor, coupled to a display, the processor in data communication with the memory and configured to execute the executable instructions to identify an insight event based on the measured analyte data within a predetermined time period, wherein the predetermined time period is between 5 minutes and 3 days; generate an insight notification based on the insight event, the insight event including one of an analyte rapidly rising insight event, an analyte spike insight event, an analyte falling insight event, an analyte back-in-range insight event, and an analyte time-in-range insight event; and present, on the display, the insight notification to the user in a graphical user interface (GUI).

Clause 10: The display device according to Clause 9, wherein the insight event is the analyte rapidly rising insight event; and the insight notification is an analyte rapidly rising insight notification that includes a graph of analyte concentration level over time, and an alphanumeric text block indicating that the analyte concentration level is rising.

Clause 11: The display device according to Clause 9, wherein the insight event is the analyte spike insight event; and the insight notification is an analyte spike insight notification that includes a graph of analyte concentration level over time, and an alphanumeric text block indicating that the analyte concentration level is rising.

Clause 12: The display device according to Clause 9, wherein the insight event is the analyte back-in-range insight event; and the insight notification is an analyte back-in-range insight notification that includes a graph of analyte concentration level over time, and an alphanumeric text block indicating that the analyte concentration level is less than an upper range threshold level and greater than a lower range threshold level.

Clause 13: The display device according to Clauses 9, 10, 11, or 12, wherein the processor is further configured to determine an occurrence of a related event, within the predetermined time period, based on related event data received from the user; the related event comprising a meal, an activity, or a medicament dosing; and the related event data including an event type, a date, and a time.

Clause 14: The display device according to Clause 13, wherein the processor is further configured to generate a related event notification based on the related event, the related event notification including a graph of analyte concentration level over time, an icon displayed at a related event start time, and an alphanumeric text block describing the related event; and present the related event notification to the user in the GUI, wherein when the related event is a meal, the icon is a meal icon and the alphanumeric text block describes a content of the meal, wherein when the related event is an activity, the icon is an activity icon and the alphanumeric text block describes the activity, and wherein when the related event is a medicament dosing, the icon is a medicament dosing icon and the alphanumeric text block describes the medicament dosing.

Clause 15: The display device according to any of Clauses 9 to 14, wherein the predetermined time period is between 5 minutes and 30 minutes, 30 minutes and 3 hours, or 3 hours and 3 days; the measured analyte data are measured glucose data; and the insight notification comprises one or more images, numbers, alphanumeric text, or graphical control elements.

The many features and advantages of disclosure are apparent from detailed specification, and, thus, it is intended by appended claims to cover all such features and advantages of disclosure which fall within scope of disclosure. Further, since numerous modifications and variations will readily occur to those skilled in art, it is not desired to limit disclosure to exact construction and operation illustrated and described, and, accordingly, all suitable modifications and equivalents may be resorted to that fall within scope of disclosure.

What is claimed is:

1. A method for providing insight notifications on a display device, the method comprising:
receiving measured analyte data from a continuous analyte monitoring sensor device worn by a user;
identifying an insight event based on the measured analyte data within a predetermined time period;
generating an insight notification based on the insight event; and
presenting the insight notification to the user in a graphical user interface (GUI).

2. The method according to claim 1, wherein the insight event comprises:
an analyte rapidly rising insight event;
an analyte spike insight event;
an analyte falling insight event;
an analyte back-in-range insight event; or
an analyte time-in-range insight event.

3. The method according to claim 2, wherein:
the insight event is an analyte rapidly rising insight event; and
the insight notification is an analyte rapidly rising insight notification that includes a graph of analyte concentration level over time, and an alphanumeric text block indicating that the analyte concentration level is rising.

4. The method according to claim 2, wherein the method further comprises:
determining an occurrence of a related event within the predetermined time period, the related event comprising a meal, an activity, or a medicament dosing.

5. The method according to claim 4, wherein determining the occurrence of the related event is based on related event data received from the user, the related event data including an event type, a date, and a time.

6. The method according to claim 5, further comprising:
generating a related event notification based on the related event, the related event notification including:
a graph of analyte concentration levels over time,
an icon displayed at a related event start time, and
an alphanumeric text block describing the related event; and
presenting the related event notification to the user in the GUI,

7. The method according to claim 6, wherein:
the related event is a meal, the icon is a meal icon, and the alphanumeric text block describes a content of the meal;
the related event is an activity, the icon is an activity icon, and the alphanumeric text block describes the activity; or
the related event is a medicament dosing, the icon is a medicament dosing icon, and the alphanumeric text block describes the medicament dosing.

8. The method according to claim 1, wherein:
the measured analyte data are measured glucose data;
the insight notification comprises one or more images, numbers, alphanumeric text, or graphical control elements; and
the predetermined time period is between 5 minutes and 30 minutes, between 30 minutes and 3 hours, or between 3 hours day and 3 days.

9. A display device, comprising:
a wireless transceiver configured to receive measured analyte data from a continuous analyte monitoring sensor device worn by a user;
a memory comprising executable instructions; and
a processor, coupled to a display, the processor in data communication with the memory and configured to execute the executable instructions to:
identify an insight event based on the measured analyte data within a predetermined time period;
generate an insight notification based on the insight event; and
present, on the display, the insight notification to the user in a graphical user interface (GUI).

10. The display device according to claim 9, wherein the insight event comprises:
an analyte rapidly rising insight event;
an analyte spike insight event;
an analyte falling insight event;
an analyte back-in-range insight event; or
an analyte time-in-range insight event.

11. The display device according to claim 10, wherein:
the insight event is the analyte rapidly rising insight event; and
the insight notification is an analyte rapidly rising insight notification that includes a graph of analyte concentration level over time, and an alphanumeric text block indicating that the analyte concentration level is rising.

12. The display device according to claim 10, wherein the processor is further configured to determine an occurrence of a related event within the predetermined time period, the related event comprising a meal, an activity, or a medicament dosing.

13. The display device according to claim 12, wherein the processor is further configured to determine the occurrence of the related event based on related event data received from the user, the related event data including an event type, a date, and a time.

14. The display device according to claim 13, wherein the processor is further configured to:
generate a related event notification based on the related event, the related event notification including:
a graph of analyte concentration level over time,
an icon displayed at a related event start time, and
an alphanumeric text block describing the related event; and
present the related event notification to the user in the GUI,
wherein:
the related event is a meal, the icon is a meal icon, and the alphanumeric text block describes a content of the meal,
the related event is an activity, the icon is an activity icon, and the alphanumeric text block describes the activity, or
the related event is a medicament dosing, the icon is a medicament dosing icon, and the alphanumeric text block describes the medicament dosing.

15. The display device according to claim 9, wherein:
the measured analyte data are measured glucose data;
the insight notification comprises one or more images, numbers, alphanumeric text, or graphical control elements; and
the predetermined time period is between 5 minutes and 30 minutes, between 30 minutes and 3 hours, or between 3 hours day and 3 days.

16. A continuous analyte monitoring (CAM) system, comprising:
a sensor device worn by a user, the sensor device including:
an analyte sensor configured to measure an analyte concentration levels,
a processor configured to generate measured analyte data based on the measured analyte concentration levels, and
a wireless transceiver configured to transmit the measured analyte data; and
a display device including:
a wireless transceiver configured to receive the measured analyte data, and
a processor, coupled to a display, configured to:
identify an insight event based on the measured analyte data within a predetermined time period,
generate an insight notification based on the insight event, and
present the insight notification to the user in a graphical user interface (GUI).

17. The CAM system according to claim 16, wherein the insight event comprises:
an analyte rapidly rising insight event;
an analyte spike insight event;
an analyte falling insight event;
an analyte back-in-range insight event; or
an analyte time-in-range insight event.

18. The CAM system according to claim 17, wherein
the insight event is an analyte rapidly rising insight event; and
the insight notification is an analyte rapidly rising insight notification that includes a graph of analyte concentration level over time, and an alphanumeric text block indicating that the analyte concentration level is rising.

19. The CAM system according to claim 17, wherein the processor of the display device is further configured to:
determine an occurrence of a related event based on related event data received from the user, the related event data including an event type, a date, and a time;
generate a related event notification based on the related event, the related event notification including:
a graph of analyte concentration level over time,
an icon displayed at a related event start time, and
an alphanumeric text block describing the related event; and
present the related event notification to the user in the GUI,
wherein:
the related event is a meal, the icon is a meal icon, and the alphanumeric text block describes a content of the meal,
the related event is an activity, the icon is an activity icon, and the alphanumeric text block describes the activity, or
the related event is a medicament dosing, the icon is a medicament dosing icon, and the alphanumeric text block describes the medicament dosing.

20. The CAM system according to claim 16, wherein:
the measured analyte data are measured glucose data;
the insight notification comprises one or more images, numbers, alphanumeric text, or graphical control elements; and
the predetermined time period is between 5 minutes and 30 minutes, between 30 minutes and 3 hours, or between 3 hours day and 3 days.
